(19) United States
(12) Patent Application Publication
(10) Pub. No.: US 2011/0236917 A1
(43) Pub. Date: Sep. 29, 2011

(54) Diagnosis of Alzheimer's Disease

(75) Inventor: Ira Leonard Goldknopf, The Woodlands, TX (US)

(73) Assignee: Power3 Medical Products, Inc., The Woodlands, TX (US)

(21) Appl. No.: 13/153,669

(22) Filed: Jun. 6, 2011

Related U.S. Application Data
(63) Continuation of application No. 12/927,343, filed on Nov. 12, 2010.
(60) Provisional application No. 61/281,478, filed on Nov. 17, 2009.

(51) Int. Cl. C12Q 1/37

(52) U.S. Cl. 435/23

(57) ABSTRACT

Embodiments of an assay for the screening, diagnosis, and differentiation of Alzheimer's disease uses selected sets of serum protein biomarkers. The assay is used to distinguish Alzheimer's disease from Parkinson's disease, other neurodegenerative diseases, and normal controls. Specific sets of serum protein biomarkers are identified for the diagnosis of Alzheimer's disease in drug naïve and drug treated patients.

### CROSS REFERENCE TO RELATED APPLICATIONS

This application is a continuation of U.S. Ser. No. 12/927,343, filed on Nov. 12, 2010, entitled “Diagnosis of Alzheimer's disease” by Ira Leonard Goldknopf, which claims priori to U.S. provisional application Ser. No. 61/281,478 filed Nov. 17, 2009, and entitled “Alzheimer's Specific Abnormal Serum Protein Concentrations for Diagnosis in the Clinical Setting.”

This application is related to U.S. Ser. No. 12/217,885 filed on Jul. 8, 2008, entitled “Multiple forms of Alzheimer's disease based on differences in concentrations of protein biomarkers in blood serum” by Ira Leonard Goldknopf, which is incorporated herein in its entirety by reference.

### FIELD OF THE INVENTION

The present invention relates in general to a method for screening and/or diagnosing Alzheimer's disease. In particular, the present invention relates a set of biomarkers and their use in the screening and/or diagnosis of Alzheimer's disease in humans.

### DESCRIPTION OF THE RELATED ART

Alzheimer's disease (AD) is a progressive brain disorder that gradually destroys a person's memory and ability to learn, reason, make judgments, communicate and carry out daily activities. As AD progresses, individuals may also experience changes in personality and behavior, such as anxiety, suspiciousness or agitation, as well as delusions or hallucinations. An estimated 4.5 million Americans have AD. The number of Americans with AD has more than doubled since 1980 and is predicted to reach from 11.3 million to 16 million patients.

Presently, the diagnosis of AD is a clinical one. The usual diagnostic process consists of a full medical history, a comprehensive physical and neurological examination, as well as assessing a patient's cognitive status. Cognitive impairment is typically tested using standardized cognitive screening test call the Mini Mental State Examination (MMSE). A patient's MMSE score is generally combined with clinical features and laboratory test results to classify the severity of AD as Normal, Mild, Moderate, or Severe.

Detection of abnormalities in the genome of an individual can reveal the risk or potential risk for individuals to develop a disease. The transition from risk to emergence of disease can be characterized as an expression of genomic abnormalities in the proteome. Thus, the appearance of abnormalities in the proteome signals the beginning of the process of cascading effects that can result in the deterioration of the health of the patient. Therefore, detection of proteomic abnormalities at an early stage is desired in order to allow for detection of disease either before it is established or in its earliest stages where treatment may be effective.

Accurate and specific diagnosis of Alzheimer's disease (AD) can be difficult, especially in early stages when there is often an insidious onset of symptoms overlapping with different disorders. Delays in diagnosis or inappropriate treatment can allow irreversible neurological damage and/or render treatment less effective, than if people were diagnosed and treated earlier, particularly on the basis of an accurate blood test that could be performed on a routine basis.

With the advent of FDA approved drugs for the symptoms of dementia, patients with suspected dementias are often given medication early, prior to diagnosis, and they respond with improved cognition. This, along with widespread use of other over the counter and prescription medications in general introduces additional variables that must be taken into account when attempting to arrive at an accurate diagnosis. Employing profiles of gene expression or protein biomarker concentrations for molecular diagnostics must be carried out with this in mind. Hence, a desired attribute of a diagnostic test for Alzheimer's disease is the ability to diagnose AD in drug treated patients.

In Alzheimer's disease, the dementia risk Apo E ε4 gene allele is inherited as one of three Apo E alleles, termed ε2, ε3, and ε4, with mean frequencies in the general population of about 8%, 78%, and 14%, respectively (Utermann G. et al. 1980. Am. J. Hum. Genet. 32, 339-347), whereas approximately 50% of the Alzheimer's disease patients are Apo E ε4 allele carriers. The degree of risk of dementia conferred by the Apo E ε4 allele rises in a “gene dose” dependent manner (Corder E. H. et al. 1993. Science 261, 921-923), increasing with the number of Apo E ε4 alleles inherited from zero (i.e. Apo E ε4 non-carriers), to carriers of one Apo E ε4 allele (i.e. ε4/ε3; ε4/ε2 hetero-zygotes), to two Apo E ε4 alleles (i.e. ε4/ε4 homo-zygotes, all of whom are capable of developing Alzheimer's disease, although those lacking the Apo E ε4 allele may tend to get the disease at a later age of onset. Furthermore, the parenchymal and vascular amyloid neuropathology is greater in non-demented Apo E ε4 carriers than in non-demented non-carriers of Apo E ε4. Therefore, another desired attribute of a diagnostic test for Alzheimer's disease is the ability to diagnose AD in patients vs. normal and disease controls in Apo E ε4 allele carriers and non-carriers.

Two-dimensional gel electrophoresis has been used in research laboratories for biomarker discovery for three decades. In the past, this method has been considered highly specialized, labor intensive and non-reproducible. Recently with the advent of integrated supplies, robotics, and software, combined with bioinformatics, the progression of this proteomics technique in the direction of diagnostics has become feasible.

The utility of 2D gel electrophoresis is based on its ability to detect changes in expression of intact proteins to separate and discriminate between specific intact protein isoforms that arise due to variations in amino acid sequence and/or post-synthetic protein modifications such as phosphotylation, ubiquitination, conjugation with ubiquitin-like proteins, acetylation, glycosylation, and proteolytic processing. These are critical features in cellular and physiological regulation.

Recent progress using a novel form of mass spectrometry called surface enhanced laser desorption and ionization time of flight (SELDI-TOF) for the testing of ovarian cancer and Alzheimer's disease has led to an increased interest in proteomics as a diagnostic tool (Petrocoin, E. F. et al. 2002. Lancet 359:572-577; Lewczuk, P. et al. 2004. Biol. Psychiatry 55:524-530). Furthermore, proteomics has been applied to the study of breast cancer through use of 2D gel electrophoresis and image analysis to study the development and progression of breast carcinoma in patients and in plasma from Alzheimer's disease patients (Kuerer, H. M. et al. 2002. Cancer 95:2276-2282; Ueno, I. et al. 2000. Electrophoresis 21:1832-1845). In the case of breast cancer, breast ductal fluid specimens were used to identify distinct protein expression patterns in bilateral matched pair ductal fluid samples of women with unilateral invasive breast carcinoma.

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to raise protective antibodies or in a kit to identify the presence or absence of the biomarker in individuals who may have been exposed to mustard poisoning.

There is a tremendous need for a simple assay that can be used to screen patients for Alzheimer's disease (AD), as well as a definitive diagnostic test to confirm the diagnosis of AD and distinguish it from other AD-Like disorders that display similar symptoms but have different treatment options and prognosis. Clinicians have long sought such tests in hopes of providing earlier treatment decisions and improved patient outcomes.

### SUMMARY OF THE INVENTION

The present invention is a diagnostic assay for differentiating patient's having Alzheimer's disease (AD) from patients with AD-Like disorders, and from non-demented normal controls. The method comprises collecting a biological sample from a patient having symptoms consistent with AD, quantitating up to 57 protein biomarkers identified as related to AD or AD-Like disorders, and determining whether or not the patient has AD or an AD-Like disorder based on the statistical analysis of the quantity of the selected protein biomarkers.

One aspect of the present invention is a method for screening a patient for AD or AD-Like disorders. The method includes: collecting a serum sample from a patient having symptoms consistent with AD, separating the proteins in the serum sample by 2D gel electrophoresis, quantitating a panel of protein biomarkers, and determining whether or not the patient has a AD or an AD-Like disorder based on the quantity of those biomarkers in the patient's serum.

Another aspect of the present invention is a method for diagnosing Alzheimer's disease comprising: collecting a serum sample from a test subject that is untreated with anti-dementia drugs; analyzing the serum sample for a change in expression of a set of forty seven protein biomarkers; and using the change in expression of the set of biomarkers to diagnose the test subject, wherein the set of biomarkers includes Apolipoprotein E4, Apolipoprotein E3, and a Transthyretin protein, a Haptoglobin protein; a Complement C3 protein, a Complement Factor, and a Transferrin.

Yet another aspect of the present invention is a method for diagnosing Alzheimer's disease comprising: obtaining a patient serum sample from a test subject being treated with an anti-dementia drug; determining if the patient serum sample contains Apolipoprotein E4 protein; and quantitating a concentration of a set of 57 serum proteins in the patient serum sample; whereby a variation in the serum concentration of the set of fifty seven serum proteins from a mean serum concentration of the set of 57 serum protein in non-Alzheimer control serum is a positive diagnosis of Alzheimer's disease.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is a digital fluorescent image of blood serum proteins resolved by 2-dimensional polyacrylamide gen electrophoresis with 57 biomarkers circled and numbered.

FIG. 2 shows the serum concentration as the median fold of mean standard normal concentration of 47 biomarkers in age-matched normal control individuals and drug naïve Alzheimer's patients.

FIG. 3 shows the serum concentration as the median fold of mean standard normal concentration of 47 biomarkers in age-matched normal control individuals, drug naïve Alzheimer's patients, and drug naïve patients having AD-like symptoms.

FIG. 4 shows the serum concentration as the median fold of mean standard normal concentration of 47 biomarkers in age-matched normal control individuals, drug naïve Alzheimer's patients, drug naïve patients having AD-like symptoms, and drug anive AD-like Parkinson's disease patients.

FIG. 5 shows the serum concentration as the median fold of mean standard normal concentration of 47 biomarkers in age-matched normal control individuals, drug naïve Alzheimer's patients, and drug naïve patients with Lewy body dementia having AD-like symptoms.

FIG. 6 shows the serum concentration as the median fold of mean standard normal concentration of 47 biomarkers in age-matched normal control individuals, drug naïve Alzheimer's patients, and drug naïve stroke related ±mixed AD-like dementia.

FIG. 7 shows the serum concentration as the median fold of mean standard normal concentration of 47 biomarkers in age-matched normal control individuals, drug nave Alzheimer's patients, and drug naïve Frontotemporal ±mixed AD-like dementia.

FIGS. 8A and 8B show the discriminate analysis of serum samples from age-matched normal control individuals, drug naïve Alzheimer's patients, and drug naïve patients with AD-like disorders.

FIG. 9 shows the serum concentration as the median fold of mean standard normal concentration of 47 biomarkers in age-matched normal control individuals, drug naïve Alzheimer's patients, and drug treated Alzheimer's patients.

FIG. 10 shows the medication profile of patients from the prospectively collected newly drawn AD patient samples (“drug treated Alzheimer's disease patients,” DTAD, n=39).

FIG. 11 shows the multivariate linear discriminate analysis and receiver operator characteristics of the probabilities of diagnosis obtained employing the combined concentrations of the 47 biomarkers to discriminate between prospectively collected and banked drug naïve AD patient samples (DNAD, n=44) and prospectively collected newly drawn drug treated AD patient samples (DTAD, n=39).

FIG. 12 shows the serum concentration as the median fold of mean standard normal concentration of 57 biomarkers in age-matched normal control individuals, drug treated Alzheimer's patients, and drug treated Parkinson's patients.

FIG. 13 is a schematic representation of the results of a stepwise linear discriminant analysis for distinguishing DTAD patients from age-matched normal controls and DTPD from age-matched normal controls.

FIG. 14 is a canonical plot using 14 biomarkers for distinguishing DTAD patients from age-matched normal controls and a summary of results using 14, 33, or 57 biomarkers for distinguishing DTAD patients from age-matched normal controls.

FIG. 15 is a canonical plot using 21 biomarkers for distinguishing DTPD patients from age-matched normal controls and a summary of results using 21 or 57 biomarkers for distinguishing DTPD patients from age-matched normal controls

FIG. 16 is a schematic representation of the results of a stepwise linear discriminant analysis for distinguishing DTPD patients from DTAD patients and a summary of results using 57 or 27 biomarkers to distinguish DTPD patients from DTAD patients.

FIG. 17 illustrates the sensitivity of distinguishing DTAD patients from age-matched normal controls using biomarkers selected by a stepwise linear discriminant analysis versus using biomarkers that were not selected by discriminant analysis.

FIG. 18 illustrates that the sensitivity of distinguishing DTAD patients from normal age-matched controls was essentially independent of the severity of the patients' symptoms.

FIG. 19 shows the multivariate linear discriminate analysis and receiver operator characteristics of the probabilities of diagnosing drug treated AD patient samples (DTAD) and drug treated PD patient samples (DTPD).

FIG. 20 shows the multivariate linear 3 way discriminate analysis and receiver operator characteristics of DTAD vs. DTPD vs. normal age-matched controls and for DTAD vs. Not DTAD, where Not DTAD includes DTPD patients and normal age-matched controls.

FIG. 21 shows a comparative statistical Dot, Box and Whiskers graph of the concentration of Apolipoprotein E4 protein in blood serum of drug treated AD patients, drug treated PD patients, and age-matched normal controls.

FIG. 22 shows the serum concentration as the median fold of mean standard normal concentration of 57 serum biomarkers in Apo E4(+) DTAD, DTPD, and age-matched normal controls.

FIG. 23 shows the serum concentration as the median fold of mean standard normal concentration of 57 serum biomarkers in Apo E4(−) DTAD, DTPD, and age-matched normal controls.

FIG. 24 shows the multivariate linear 3 way discriminate analysis and receiver operator characteristics of Apo E4(+) DTAD vs. DTPD vs. normal age-matched controls.

FIG. 25 shows the multivariate linear 3 way discriminate analysis and receiver operator characteristics of Apo E4(−) DTAD vs. DTPD vs. normal age-matched controls.

FIG. 26 is a schematic representation of one embodiment of a method for diagnosing Alzheimer's disease and a summary of results of a linear discriminate analysis of Apo E4 (+) DTAD patients vs. DTPD patients vs. age-matched normal controls, a linear discriminate analysis of Apo E4 (−) DTAD patients vs. DTPD patients vs. age-matched normal controls, and a combined 3 way linear discriminate analysis of the Apo E4(+) and ApoE4(−) DTAD vs. DTPD vs. age-matched normal controls.

### DESCRIPTION OF THE PREFERRED EMBODIMENTS

Embodiments of the present invention include screening and diagnostic tests for differentiating individuals with Alzheimer's disease (AD) in drug treated patients and in drug naïve patients. Patients with AD are differentiated from patients without AD, and from patients with AD-Like disorders that express symptoms like AD. The method is based on the use of 2-dimensional (2D) gel electrophoresis to separate the complex mixture of proteins found in blood serum and the quantitation of a group of identified biomarkers to differentiate patients having AD from patients having other AD-Like disorders.

In the context of the present invention, the “protein expression profile” corresponds to the steady state level of the various proteins in biological samples that can be expressed quantitatively.

In the context of the present invention, a “biomarker” corresponds to a protein or protein fragment present in a biological sample from a patient, wherein the quantity of the biomarker in the biological sample provides information about whether the patient exhibits an altered biological state such as AD or an AD-Like disorder.

The method of the present invention is based on the quantification of specified proteins. Preferably the proteins are separated and identified by 2D gel electrophoresis as described is U.S. Utility patent application Ser. No. 12/217,885 filed Jul. 8, 2008 and incorporated herein by reference.

2D gel electrophoresis has been used in research laboratories for biomarker discovery since the 1970's (Orrick, L. R. et al. 1973. Proc. Natl. Sci. U.S.A. 70:13 16-1320; Goldknopf, I. L. et al. 1975. J. Biol Chem. 250:71282-7187; O'Farrell, P. et al. 1975. J. Biol. Chem. May 250:4007-4021; Anderson, L. and Anderson, N. G. 1977. Proc. Natl. Acad. Sci. U.S.A. 74:5421-5425; Goldknopf, I. L. and Busch, H.1977. Proc. Natl. Acad. Sci. USA 74:864-868). In the past, this method has been considered highly specialized, labor intensive and non-reproducible.

Only recently with the advent of integrated supplies, robotics, and software combined with bioinformatics has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as phosphorylation, nitrosylation, ubiquitination, conjugation with ubiquitin-Like proteins, acetylation, and glycosylation. These are important variables in cell regulatory processes involved in disease states.

There are few comparable alternatives to 2D gels for tracking changes in protein expression patterns related to disease progression. The introduction of high sensitivity fluorescent staining, digital image processing and computerized image analysis has greatly amplified and simplified the detection of unique species and the quantification of proteins. By using known protein standards as landmarks within, each gel run, computerized analysis can detect unique differences in protein expression and modifications between two samples from the same individual or between several individuals.

Subjects

Patients and age-matched controls were from three clinical sites (1) Baylor College of Medicine, Houston, Tex., USA; (2) Banner Sun Health Research Institute, Sun City, Ariz., USA, and (3) University of Thessaly, Larissa, Greece. The number of serum samples investigated from patients and controls are listed in Table 1.

The study compared biomarker concentrations in serum samples of healthy participants and those with neurodegenerative diseases in the initial biomarker panel identification (site 1), and with healthy participants and those with AD and PD in the extended investigation of the panel (sites 2 and 3). AD and PD patients underwent clinical evaluation to provide clinical data, including the severity of AD and PD symptoms. The severity of AD was recorded according to the MMSE score, and the severity of PD was measured according to the Hoehn and Yahr Scale and the Unified Parkinson's Disease Rating Scale (UPDRS).

Inclusion and exclusion criteria for AD and PD patients are listed in Tables 2 and 3. Patient information provided included demographics and medical history. Evaluation of clinical signs of PD included rigidity (stiffness or inflexibility of limbs and joints), bradykinesia/akinesia (slowness of movement/absence of movement), tremor (involuntary, regular rhythmic shaking of the limb, the head, the mouth, the tongue or the entire body) and postural instability (coordination and impaired balance). Also evaluated were the history of past illness, patients' current health problems, and copies of conventional imaging (CAT, PET scans, MRI of brain, SPECT, etc). All forms and copies of reports were identified by study number only in order to maintain confidentiality; a copy of the above mentioned medical information was sent to the testing site in accordance with Health Information Privacy concerns.

Separation and Image Analysis of Proteins in Patient Samples

Sample preparation and electrophoresis were performed essentially as described is U.S. Utility patent application Ser. No. 12/217,885 filed Jul. 8, 2008 and incorporated in its entirety by reference. The first dimension electrophoresis (100 μg of serum proteins/gel) was on immobilized 11 cm IEF strips (Bio-Rad Laboratories, Hercules Calif.), pH 5-8, and the second dimension was on pre-cast 8-16% acrylamide gradient CRITERION SDS-gels (Bio-Rad).

TABLE 1
Prospective banked and prospective
newly drawn patients and controls

| Blood Serum | Total No. | |
|---|---|---|
| Sample Type | of Patients^{£} | Disease Status |
| Prospective Banked | 44 | Alzheimer's disease (DNAD) |
| Samples of Drug | 29 | Parkinson's disease (DNPD) |
| Naïve (DN) Patients | 24 | AD/PD-like and Mixed (DNADL)^{†} |
| and controls^{¥} | 75 | AD/PD Age-matched Normal |
| | | Controls |
| | 136 | Amyotrophic lateral sclerosis |
| | | (DNALS) |
| | 33 | ALS-like (DNALSL)^{‡} |
| | 57 | ALS Age-matched Normal Controls |
| Prospective Newly | 39 | Alzheimer's disease (DTAD) |
| Drawn Samples of | 62 | Parkinson's disease (DTPD) |
| Drug Treated (DT) | 78 | AD/PD Age-matched Normal |
| Patients and | | Controls |
| Controls^{§} | | |

^{†}AD/PD-like disorders including Frontotemporal dementia; Lewy body dementia; Vascular (Multi-infarct) dementia; Alcohol related dementia; Semantic dementia; Stroke (CVA); Post-irradiation Encephalopathy and Seizures; Vascular (Multi-infarct) parkinsonism; Multiple system atrophy; Essential tremor; Corticalbasal ganglionic degeneration; and mixed disorders including Alzheimer's disease combined with Vascular (Multi-infarct) dementia; Alzheimer's disease combined with Lewy body dementia; Parkinson's disease combined with Lewy body dementia; Alzheimer's and Parkinson's disease combined with Lewy body dementia; Frontotemporal dementia combined with Chronic inflammatory demyelinating polyneuropathy; and Thalamic CVA combined with HX of Lung CA.
^{‡}Non-ALS disorders of motor neurons, muscles, nerves, and spinal cord.
^{¥}From Houston, TX, USA.
^{§}From Thessaly, Greece and Sun City, AZ, USA
^{£}This study was approved by the Institutional Review Boards of Baylor College of Medicine and the Banner Sun Health Research Institute and the Ethics Committee of the University of Thessaly, with written informed consent. Subjects were evaluated by neurologists Katerina Markopoulou, MD, PhD, the University of Thessaly, Greece; Marwan Sabbagh, MD and Holly Shill, MD, Banner Sun Health Research Institute, Sun City, AZ, USA, and Stanley H. Appel, MD, Baylor College of Medicine, Houston, TX, USA.

TABLE 2
Inclusion and exclusion criteria for Alzheimer's disease (AD) patients and controls
for prospective clinical validation with newly drawn samples in real time.

| Inclusions: | | Exclusions: | |
|---|---|---|---|
| 1. | Age: 55-90 Both men and women in all | 1. | Significant neurologic disease other than |
| | ethnic groups | | Alzheimer's disease including Parkinson's |
| 2. | MMSE: 12-26; CDR: 0.5, 1.0 or 2.0. | | disease, multi-infarct dementia, |
| 3. | Permitted medications stable for at least 4 | | Huntington's disease, normal pressure |
| | weeks prior to screening. | | hydrocephalus, brain tumor, progressive |
| 4. | Antidepressants with no significant | | supranuclear palsy, seizure disorder, |
| | anticholinergic side effects if they are not | | subdural hematoma, multiple sclerosis, or |
| | currently depressed and no history of major | | history of significant head trauma followed |
| | depression within the past 2 years | | by persistent neurologic defaults or known |
| 5. | Estrogen replacement therapy is | | structural brain abnormalities. |
| | discouraged | 2. | Evidence of infection, infarction, or other |
| 6. | Gingko biloba is permissible, but | | focal lesions. Subjects with multiple |
| | discouraged | | lacunes or lacunes in a critical memory |
| 7. | Washout from psychoactive medication, | | structure are excluded. |
| | e.g., excluded anti-depressants, | 3. | Presence of pacemakers, aneurysm clips, |
| | neuroleptics, chronic anxiolytics or | | artificial heart valves, ear implants, metal |
| | sedative hypnotics, etc., for at least 4 weeks | | fragments or foreign objects in the eyes, |
| | prior to screening. | | skin or body. |
| 8. | Cholinesterase permitted | 4. | History of alcohol or substance abuse or |
| 9. | Good general health, no additional diseases | | dependence within the past 2 years (DSM |
| | that interfere with study. | | IV criteria). |
| 10. | Not pregnant, lactating, or of childbearing | 5. | Any significant systemic illness or unstable |
| | potential, i.e. two years post-menopausal or | | medical condition which could lead to |
| | surgically sterile. | | difficulty complying with the protocol. |
| 11. | Willing to provide serum for biomarker | 6. | Current use of specific psychoactive |
| | studies at protocol specified time points | | medications (e.g., certain antidepressants, |
| | (optional). | | neuroleptics, chronic anxiolytics or |
| 12. | Completed 6 grades of education or good | | sedative hypnotics, etc.). |
| | work history sufficient to exclude mental | | |
| | retardation. | | |
| 13. | Fluent in English or Spanish. | | |

TABLE 3
Inclusion and exclusion criteria for Parkinson's disease
(PD) patients and controls for prospective clinical
validation with newly drawn samples in real time

| Inclusions: | | Exclusions: | |
|---|---|---|---|
| 1. | Male or female >50 years old | 1. | Secondary parkinsonism |
| 2. | Fully understood informed consent | | (Non-PD) |
| 3. | Controls: | 2. | Vascular parkinsonism |
| 4. | Healthy | 3. | Encephalitis |
| 5. | No family history of Parkinson's | 4. | Exposure to neuroleptics |
| 6. | Disease subjects: | 5. | Dementia (MMSE <25) |
| 7. | At least three signs of PD | 6. | Gaze palsy |
| | a. resting tremor | 7. | Amyotrophy |
| | b. bradykinesia | 8. | Cerebellar signs |
| | c. rigidity | 9. | Symptomatic orthostatic |
| | d. postural instability | | hypotension (mean arterial |
| 8. | Responsive to levodopa or | | pressure drop >20 mm Hg, |
| | dopamine agonists | | recumbent to standing) |
| | | 10. | Unstable medical condition |
| | | 11. | History of alcohol or |
| | | | substance abuse |
| | | 12. | Major depression (Hamilton |
| | | | score >19). History |
| | | | of malignant melanoma. |

The gels were stained and analyzed essentially as described is U.S. Utility patent application Ser. No. 12/217,885 filed Jul. 8, 2008 and incorporated in its entirety by reference. Basically, the gels were stained with SyproRuby™ (Bio-Rad Laboratories) and the fluorescent digital images of the gels were captured (FLA 7000 Imager Fujifilm; FX Imager, Bio-Rad Laboratories), and protein spot detection and quantitation performed (PDQUESTTM, Bio-Rad Laboratories). Spot quantities in parts per million (PPM) fluorescent pixel spot density were normalized to total gel density. Each serum sample was analyzed in duplicate or triplicate. Quantitation of individual spots was validated for linearity, dynamic range, limit of detection (LOD=0.66 μg/ml of serum), limit of quantify ability (LOQ=6.6 μg/ml of serum), reproducibility, and robusmess (CV≧20%).

The 2D gel patterns of age-matched normal controls were compared with each other and with the blood serum samples from the patients with neurological disease as listed in Table 1 from three clinical sites. Specifically, prospectively banked drug naïve patients and age matched controls from site 1 were compared with each other and then further compared to the prospectively newly drawn samples of drug treated patients and controls from sites 2 and 3.

The Isolation and Identification of the Protein Spots

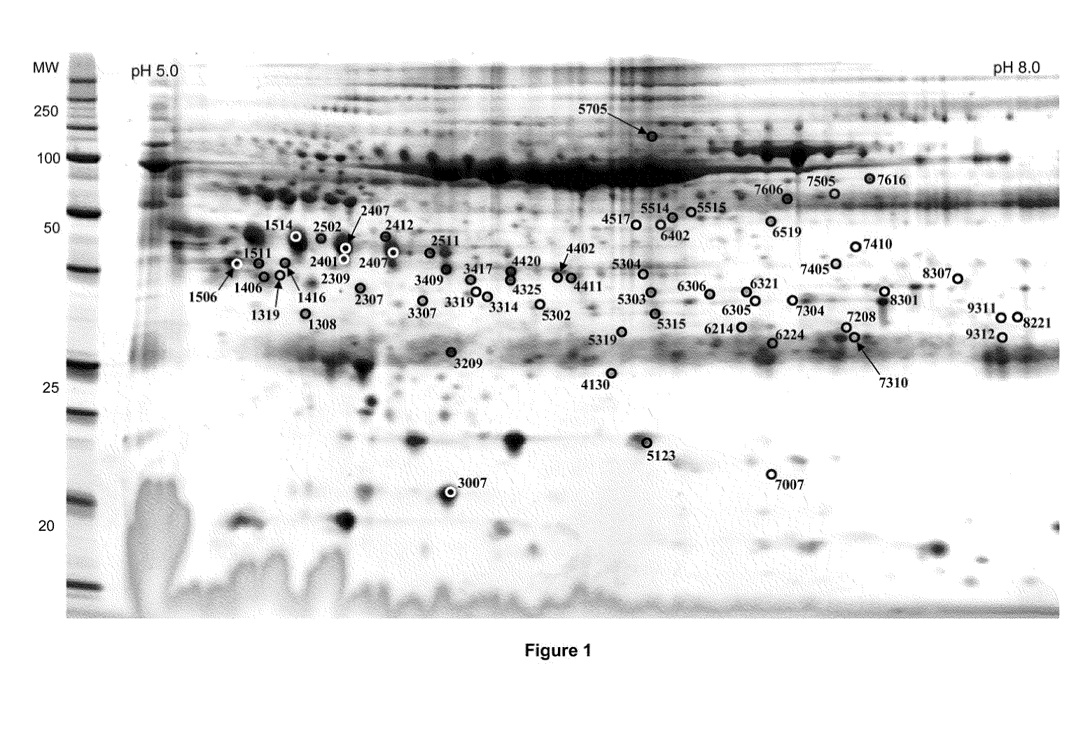

Following differential expression analysis, the blood serum concentrations of fifty seven proteins were found to be of interest (FIG. 1). These protein spots were carefully excised, in-gel digested with trypsin, and subjected to mass fingerprinting/sequence analysis by high performance liquid chromatography/tandem mass spectrometry (LC-MS/MS) as described is U.S. Utility patent application Ser. No. 12/217,885 filed Jul. 8, 2008 and incorporated herein in its entirety by reference.

Peptide fragmentation patterns were obtained from the tryptic in-gel digests of the protein spots and the patterns were subjected to public database searches using the GenBank and dbEST databases maintained by the National Center for Biotechnology Information (hereinafter referred to as the NCBI database). Those of skill in the art are familiar with searching databases, like the NCBI database. The NCBI database search results were displayed with the best matched amino acid sequences of the identified peptides and the protein accession number of the protein sequence they were derived from.

Biostatistical Analysis

Statistical significance of differences in individual biomarker blood serum concentrations (as Fold of Standard Normal Mean Concentration) between patient and controls was determined by non-parametric Dot Box and Whiskers (medians), analysis of variance, and parametric Receiver Operator Characteristics analysis using Analyze-it®″ (Analyse-it Software, Ltd., United Kingdom) imbedded in Microsoft XL. Analysis of joint performance of groups of biomarkers was by multivariate linear discriminant analysis using SAS® statistical software as described is U.S. Utility patent application Ser. No. 12/217,885 filed Jul. 8, 2008 and incorporated in its entirety by reference.

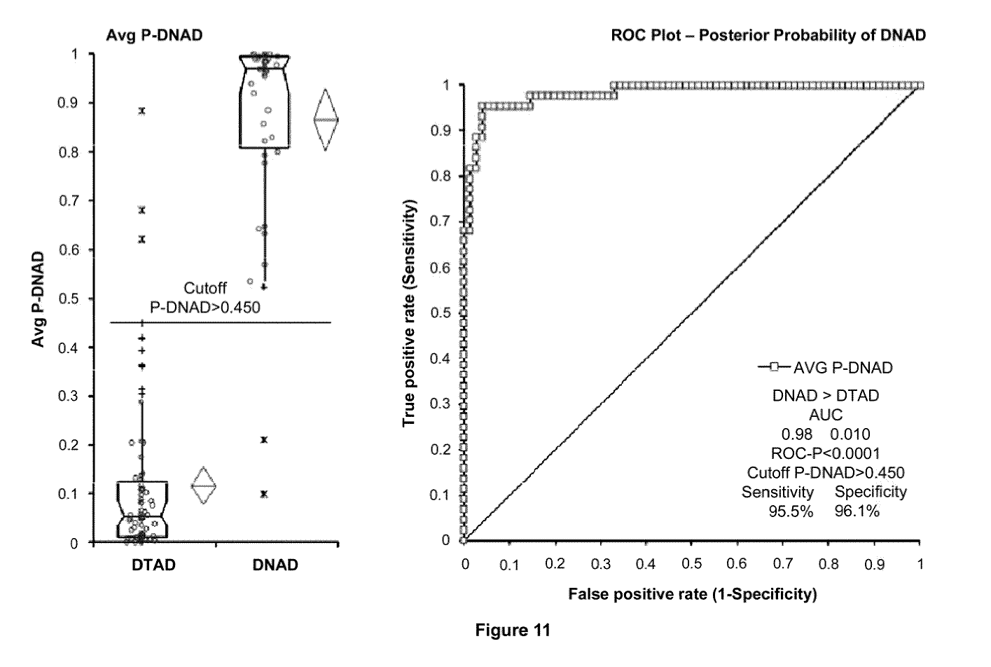

Box and Whisker plots (such as FIG. 11) give a visual representation of non-parametric descriptive statistics. The central “box” represents the distance between the first and third quartiles (inter quartile range or IQR), with the median marked as the horizontal line inside the box. The notch in the box represent the 95% confidence interval around the median (the 50th percentile); thus groups that display non-overlapping notches can be considered statistically different (p<0.05). The minimum value is the origin of the leading “whisker” and the maximum value is the limit of the trailing “whisker”. All values are plotted individually (Dots) and those values outside the whiskers are considered possible outliers, presented either as circle (far outlier) or plus sign (near outliers).

The diagnostic performance of a test or the accuracy of a test to discriminate diseased cases from normal cases is evaluated using Receiver Operating Characteristic (ROC) curve analysis. ROC curves can also be used to compare the diagnostic performance of two or more laboratory or diagnostic tests. In an ROC curve the true positive rate (Sensitivity) is plotted in function of the false positive rate (1—Specificity) for different cut-off points. Each point on the ROC plot represents a sensitivity/specificity pair corresponding to a particular decision threshold. A test with perfect discrimination (no overlap in the two distributions) has a ROC plot that passes through the upper left corner (100% sensitivity, 100% specificity). Therefore the closer the ROC plot is to the upper left corner, the higher the overall accuracy of the test.

Results

Drug Naïve Alzheimer's Disease Patient Samples. Alzheimer's Disease-Like Patients, and Age-Matched Normal Controls

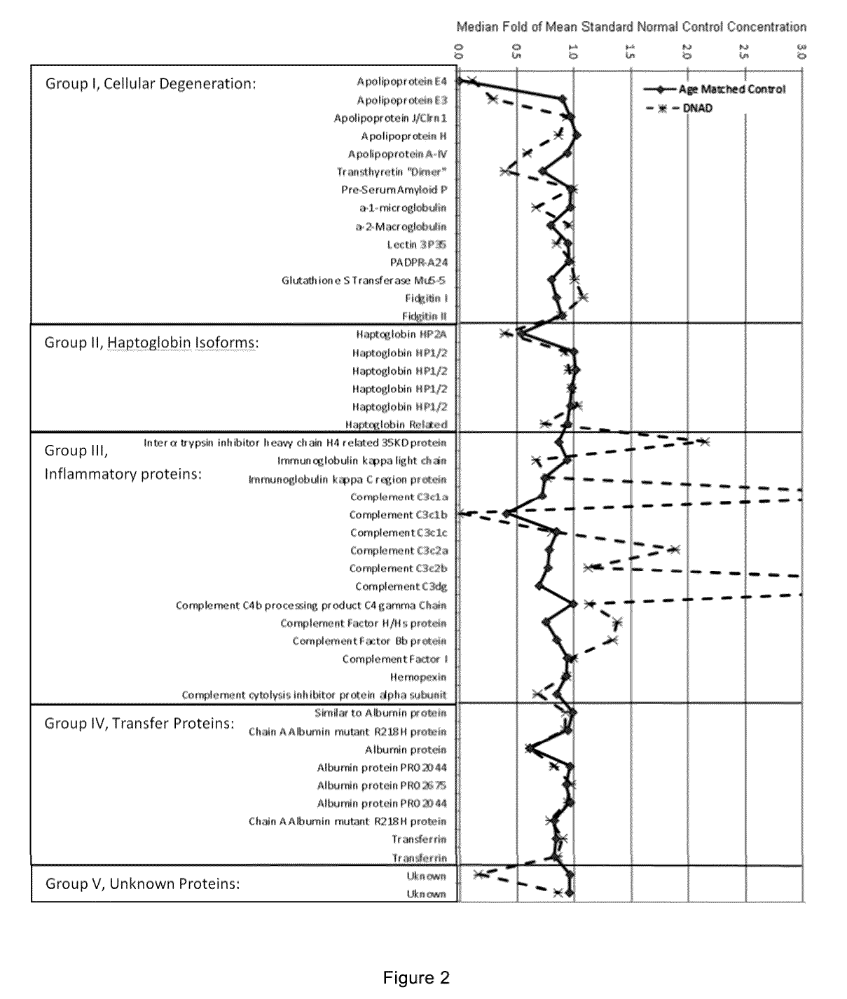

Significantly different serum concentration profiles were obtained with 47 serum protein biomarkers when prospectively collected banked serum samples from 44 drug naïve patients with Alzheimer's disease (DNAD) were compared to 151 age-matched normal controls (FIG. 2).

The serum protein biomarker spots monitored in this analysis were selected by exhaustive and painstaking comparisons of quantitative 2D gel images like that shown in FIG. 1. After the examination of the patient/control 2D gels and a biostatistical analysis of the concentrations of selected protein spots (e.g., the median fold of standard average normal PPM pixel spot densities), forty seven biomarkers were selected for discriminating drug naïve neurodegenerative disease patients and controls.

The selected proteins exhibited reproducible statistically significant disease specific abnormal serum concentrations, measured as differences between different neurodegenerative disease patient groups and normal and disease controls as described is U.S. Utility patent application Ser. No. 12/217,885 filed Jul. 8, 2008 and incorporated herein by reference.

The individual protein molecular entities resolved as spots were identified and characterized by peptide LC MS/MS profiles of spot in-gel trypsin digests and the amino acid sequence spans of the identified tryptic peptides were best-fit matched to the apparent protein spot molecular weights and isoelectric points from the 2D gels, and in some, cases, N-terminal sequencing was also performed.

The identified biomarker proteins were clustered by function into five groups: Group I, Cellular degeneration related proteins; Group II, Haptoglobin protein isoforms involved in extracellular oxidative stress; Group III, Cellular and humeral inflammatory proteins; Group IV, Transport proteins; and Group V, unknown function (see Table 4).

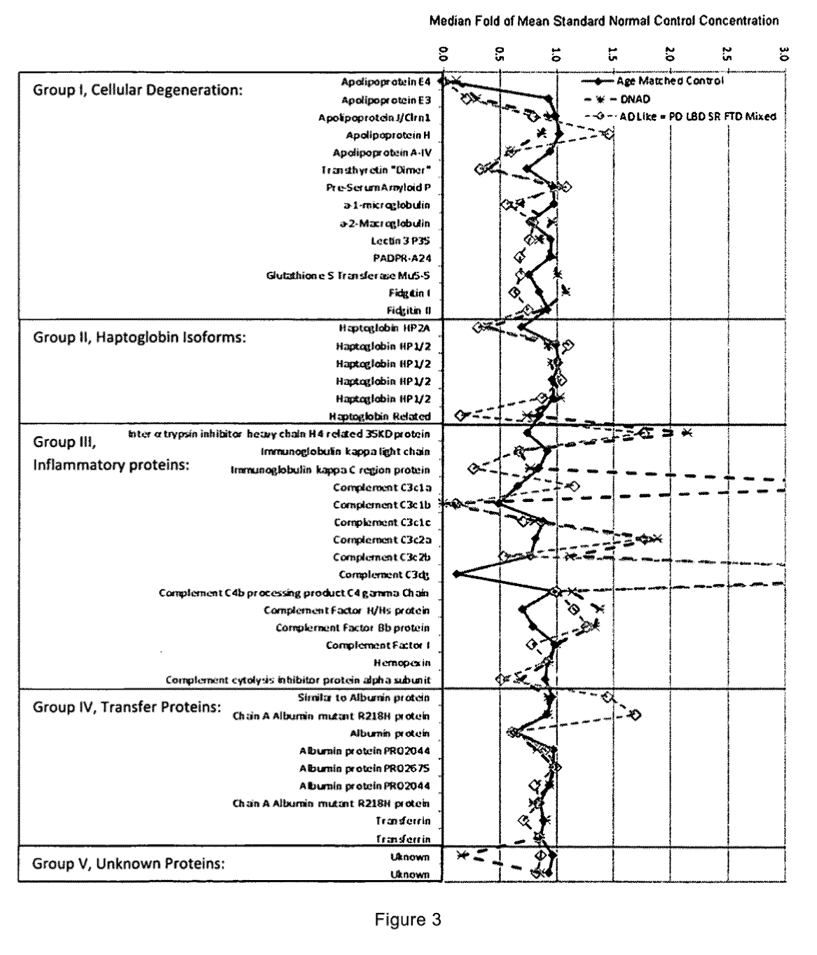

As show in FIG. 2, the serum protein concentration profile of the 44 drug naïve Alzheimer's disease patients diverged significantly from that of the 151 age-matched normal controls. When multivariate statistics on the concentrations of the 47 proteins were obtained by linear discriminant analysis as shown in FIG. 3, a 3-way discrimination was obtained between 44 patients with drug nave Alzheimer's disease vs. 26 patients with drug naïve non-AD AD-like disorders exhibiting Alzheimer's disease-like symptoms (Sensitivity=93.2%). The non-AD AD-like disorders included 12 AD-like PD patients, 7 Lewy body and mixed Lewy body dementia patients, 4 stroke-related and mixed stroke-related dementia patients, 3 Frontotemporal and mixed Frontotemporal dementia patients. The combined discrimination of AD patients from non-AD samples had a specificity of 92.2%, where Not AD included the non-AD AD-like disorders and the 151 age-matched normal controls.

Stepwise multivariate linear discriminant analysis selected 35 proteins as those providing optimal complementary discrimination between AD and Not AD (see Table 5).

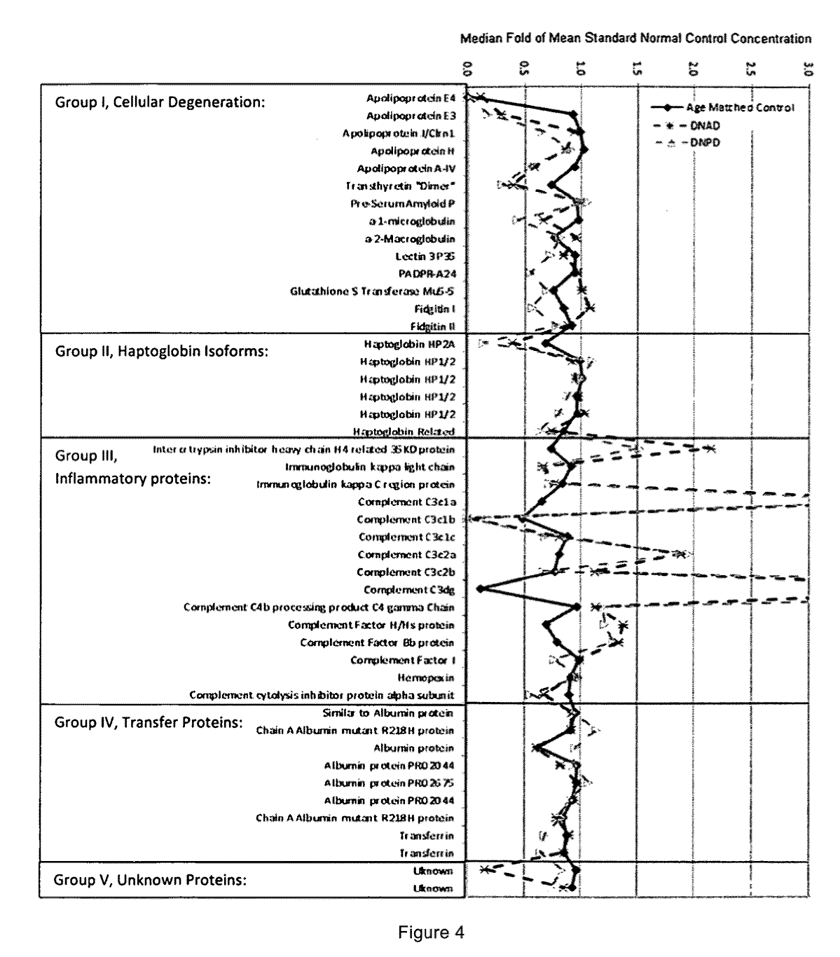

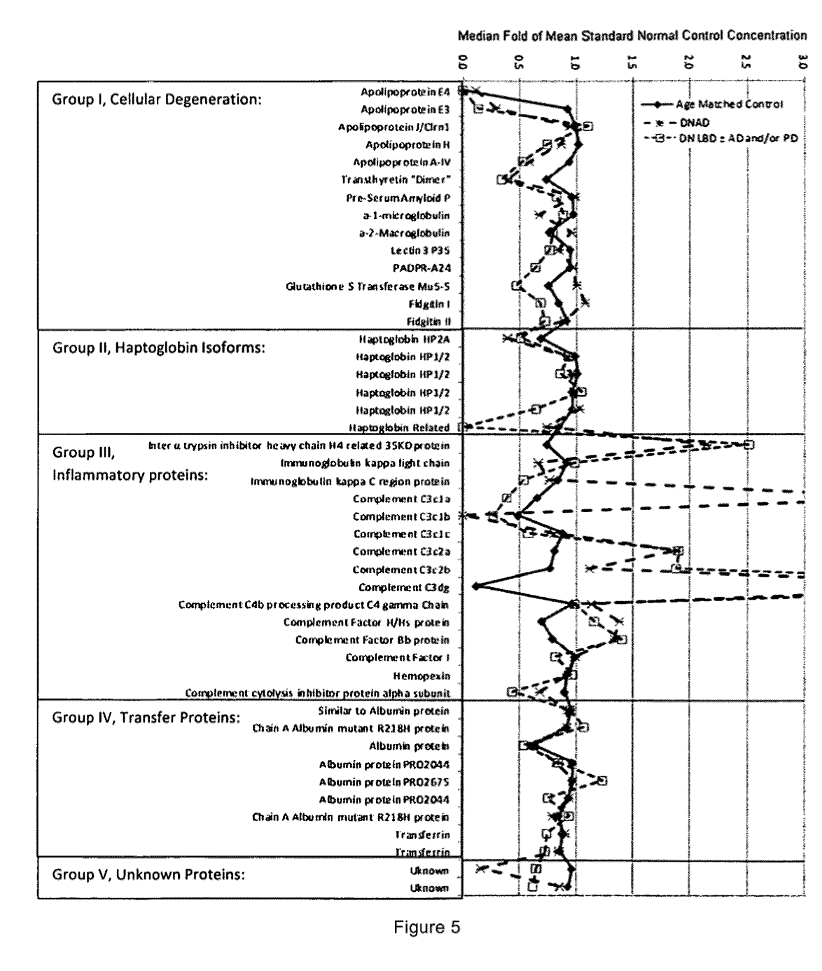

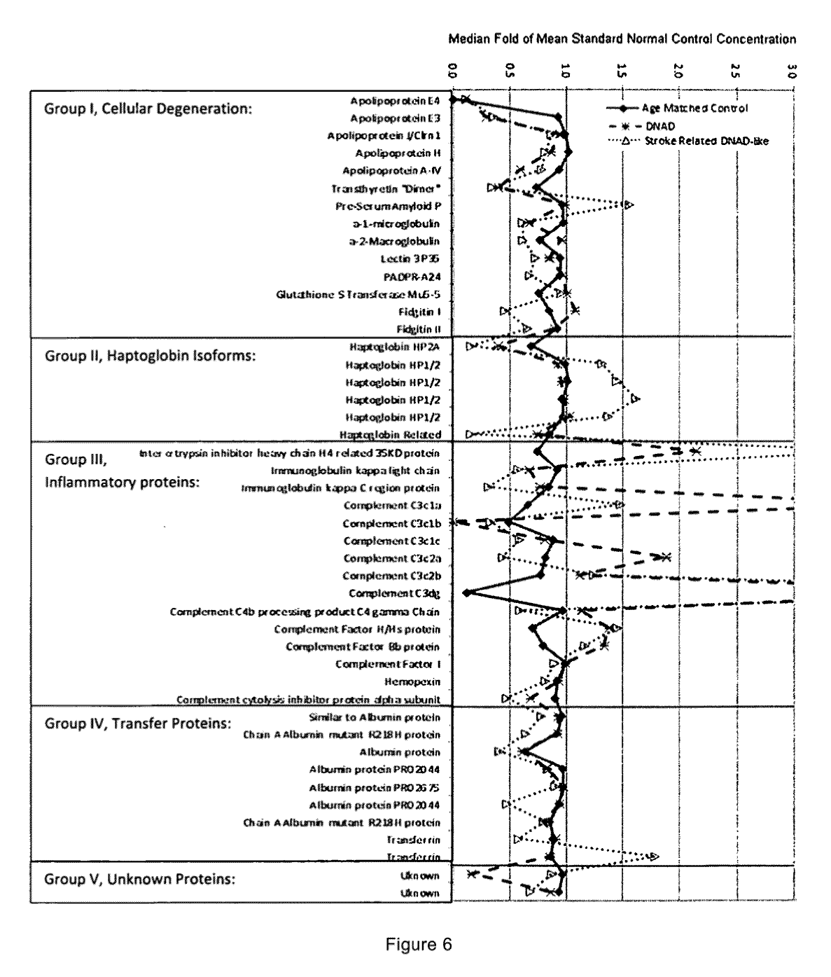

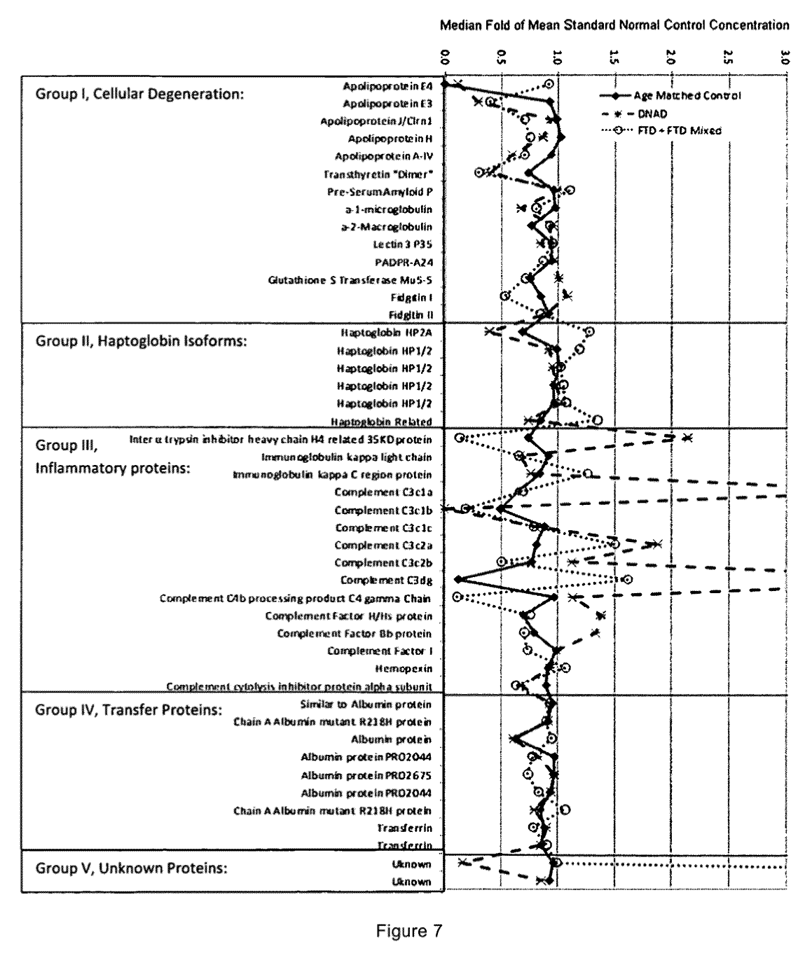

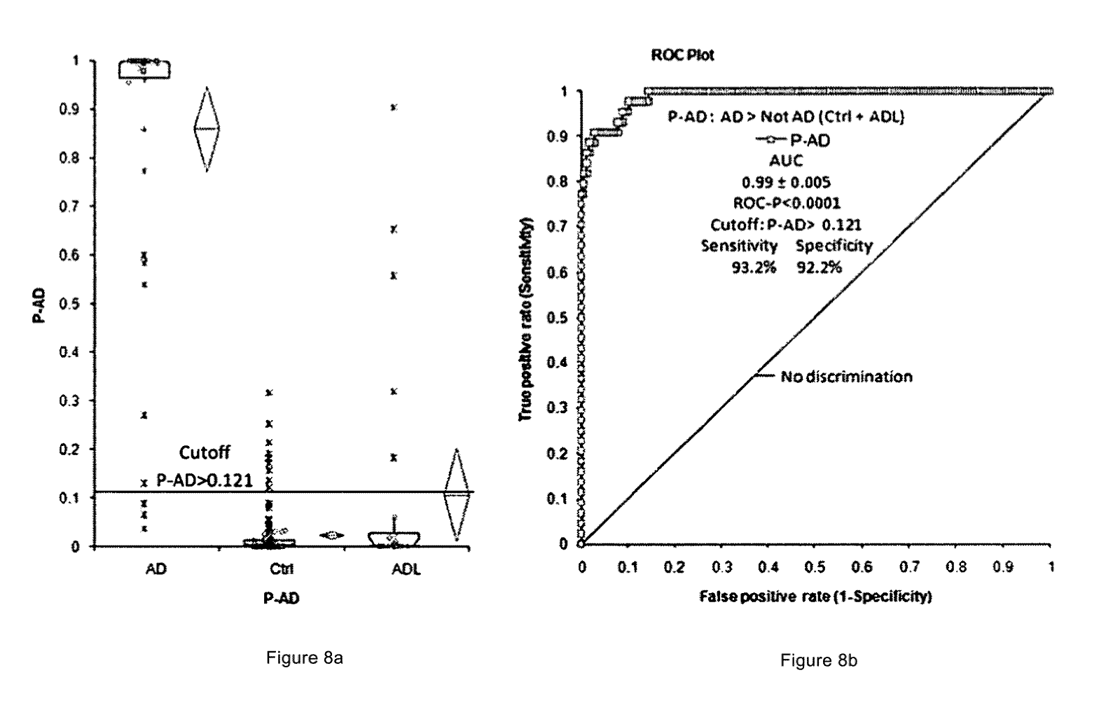

The serum protein concentration profile of the overall group of 26 AD-like patients showed significant differences from that of the 44 AD patients and the 151 age-matched normal controls (FIGS. 3 and 8), and the corresponding profiles of the AD-like subgroups were even more divergent from AD, age-matched normal controls and from each other (FIGS. 4-7).

The protein biomarkers used to discriminate between the AD patients, the AD-like patients and the normal controls include: Apolipoprotein E3, Poly-ADP-ribosyl-A24 (monoubiquitinyl-Histone H2A), Apolipoprotein E4, Apolipoprotein A-IV, Alcohol Dehydrogenase H1A3, Apolipoprotein H, Glutathione-S-Transferase Mu5-5, Clusterin 1, Fidgitin I, Fidgitin II, α-1-microglobulin, α-2-Macroglobulin, Lectin 3 P35, Transthyretin “Dimer”, Pre-serum Amyloid Protein, Haptoglobin HP-1a, Haptoglobin HP-1b, Haptoglobin HP-1c, Haptoglobin HP-1d, Haptoglobin HP-2a Protein, Haptoglobin Related Protein HP-RP, Complement C3c1a, Complement C3c1b, Complement C3c1c, Complement C3c2a, Complement C3c2b, Complement C3dg, Complement C4b Gamma Chain Protein, Complement Factor I, Complement Factor H/Hs, Complement Factor Bb, Complement Cytolysis Protein, Hemopexin, Albumin PRO2044-I, Albumen PRO2675, Inter-α-trypsin Inhibitor Heavy Chain (H4) Related 35 KD Protein, Albumin PRO2044-II, Albumin, Albumin Mutant Chain A R218H-I, Albumin Mutant Chain A R218H-II, Similar to Albumin, Transferrin I, Transferrin II, Immunoglobulin Igκ1, and Immunoglobulin Igκc.

Drug Treated Alzheimer's Disease Patient Samples, Parkinson's Disease Patients, and Age-Matched Normal Controls

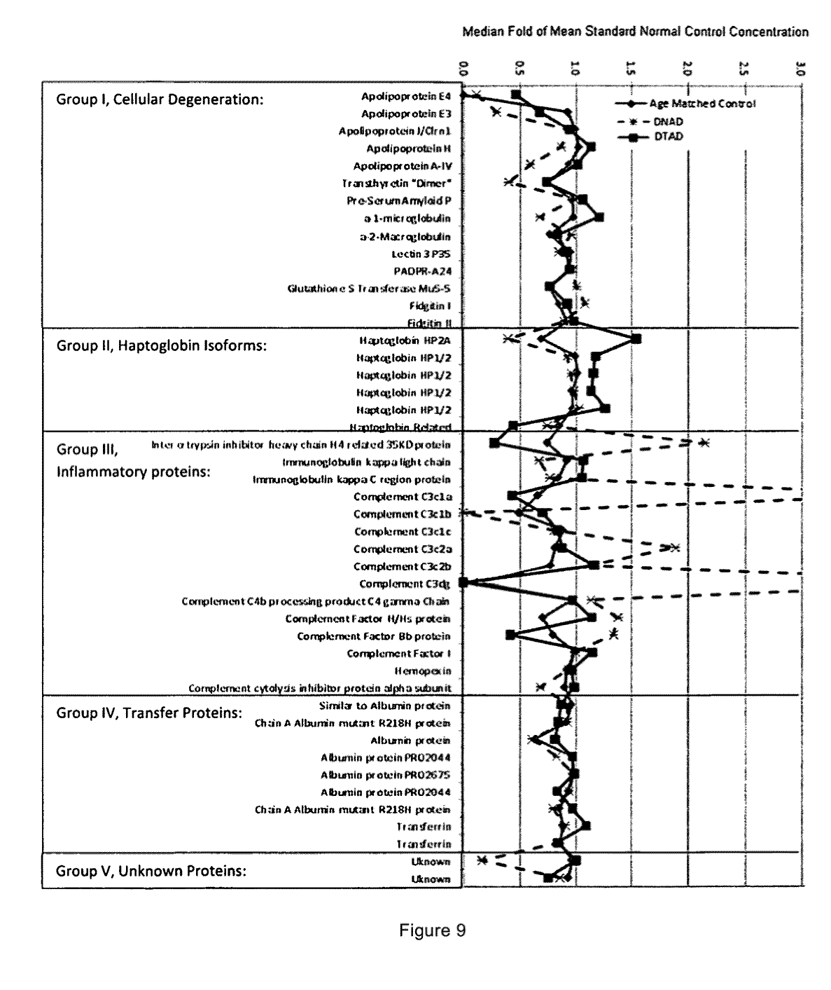

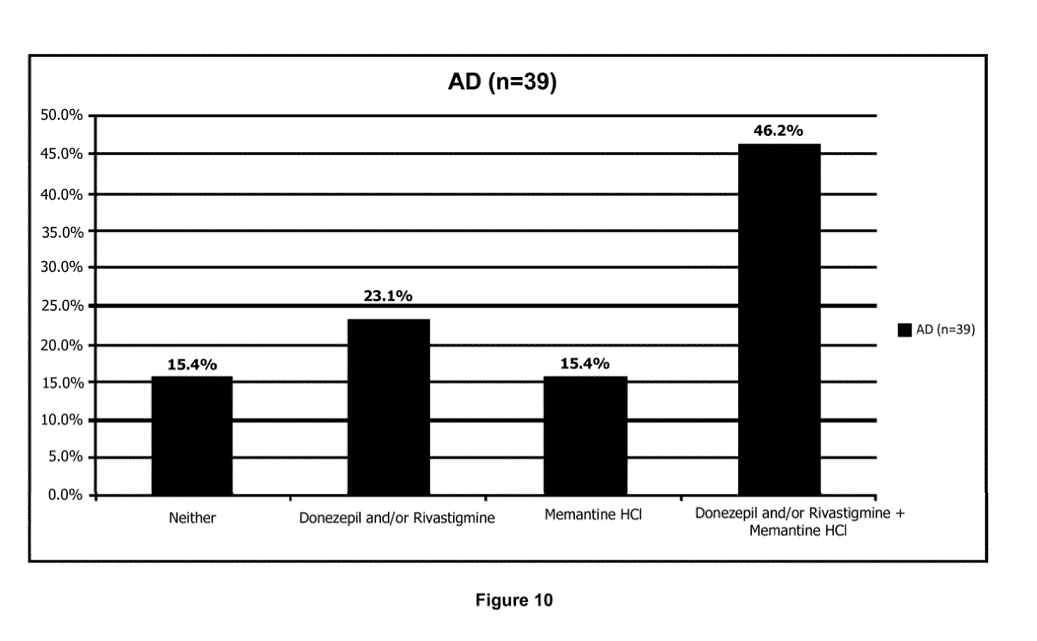

As shown in FIGS. 9 and 11, the corresponding profile for the prospectively collected newly drawn drug treated AD patient samples (DTAD, n=39) was very different from that of the prospectively collected and banked drug naïve AD patient samples (DNAD, n=44). The medication profile of the patients from the prospectively collected newly drawn AD patient samples (DTAD, n=39) is shown in FIG. 10.

TABLE 4
Protein Spot Identities:
Group I: Proteins of Cellular Degeneration

| N3314: Apolipoprotein E3 | N6306: Poly-ADP-ribosyl-A24 (monoubiquitinyl- |
|---|---|
| | Histone H2A) |
| ^{¥}N3319: Pre-Apolipoprotein E3 | ^{¥}N7007: Nucleoporin 188 |
| N5302: Apolipoprotein E4 | ^{¥}N2412: Apoptosis Inhibitor AIM-CD5 |
| N2502: Apolipoprotein A-IV | N5705: Alcohol Dehydrogenase H1A3 |
| N7606: Apolipoprotein H | N5304: Glutathione-S-Transferase Mu5-5 |
| N1406: Clusterin 1 | N8301: Fidgitin I |
| N1308: α-1-microglobulin | N6214: Fidgitin II |
| N6402: α-2-Macroglobulin | N5303: Lectin 3 P35 |
| N3307: Transthyretin “Dimer” | N3209: Pre-serum Amyloid Protein |

^{¥}N3007 Transthyretin Huntingtin Interacting Protein E

Group II: Haptoglobin Proteins
Haptoglobin Proteins:

| | |
|---|---|
| N1514: HP-1a | N2401: HP-1b |
| N2407: HP-1c | N3409: HP-1d |
| ^{¥}N2309: HP-1e | |

N5123: Haptoglobin HP-2a Protein
N4402: Haptoglobin Related Protein HP-RP

Group III: Inflammatory Proteins
Complement C3 Proteins:

| | | |
|---|---|---|
| N7310: C3c1a | N7208: C3c1b | N7410: C3c1c |
| N9311: C3c2a | N9312: C3c2b | N1511: C3dg |

N7304: Complement C4b Gamma Chain Protein

Complement Factors:

| | |
|---|---|
| N1416: I N4411: H/Hs | N7616: Bb |

N1506: Complement Cytolysis Protein

N6519: Hemopexin

N2307: Inter-α-trypsin Inhibitor Heavy Chain (H4) Related 35 KD Protein

Immunoglobulins:

| | |
|---|---|
| N6224: Igκl | N5319: Igκc |

Group IV: Transfer proteins

| | |
|---|---|
| N2511: ALB PRO2044-I | N3417: ALB PRO2675 |
| N4325: ALB PRO2044-II | N4517: Alb Protein |

N5514: Mutant Chain A R218H-I
N4420: Mutant Chain A R218H-II

N5315: Similar to Albumin
N7405: Transferrin I
N8307: Transferrin II

Group V: Function Unknown

| | | |
|---|---|---|
| N4130: X₂ | ^{¥}N5515: X₁ | ^{¥}N1319: X₆ |
| N6321: X₅ | ^{¥}N7505: X₇ | ^{¥}N8221: X₈ |

^{¥}10 additional protein spots, Bio-mined for DTAD and DTPD

TABLE 5
This analysis uses 47 biomarkers, ad vs adl vs control
The STEPDISC Procedure
Stepwise Selection Summary

| | | | | | | | Average | |
|---|---|---|---|---|---|---|---|---|
| | | | | | | | Squared | |
| | | Partial | | | Wilks' | Pr < | Canonical | Pr > |
| Step | Label | R-Square | F Value | Pr > F | Lambda | Lambda | Correlation | ASCC |
| 1 | N4130 | 0.3781 | 183.88 | <.0001 | 0.621937 | <.0001 | 0.18903148 | <.0001 |
| 2 | N7606 | 0.351 | 163.32 | <.0001 | 0.403644 | <.0001 | 0.36409755 | <.0001 |
| 3 | N1511 | 0.2649 | 108.67 | <.0001 | 0.296703 | <.0001 | 0.44945096 | <.0001 |
| 4 | N3314 | 0.1762 | 64.38 | <.0001 | 0.244423 | <.0001 | 0.49575635 | <.0001 |
| 5 | N6321 | 0.1173 | 39.95 | <.0001 | 0.215742 | <.0001 | 0.52604052 | <.0001 |
| 6 | N8301 | 0.0711 | 22.95 | <.0001 | 0.200408 | <.0001 | 0.54291252 | <.0001 |
| 7 | N7310 | 0.0581 | 18.46 | <.0001 | 0.188771 | <.0001 | 0.55594225 | <.0001 |
| 8 | N7410 | 0.057 | 18.07 | <.0001 | 0.178013 | <.0001 | 0.56981004 | <.0001 |
| 9 | N6306 | 0.0428 | 13.36 | <.0001 | 0.170389 | <.0001 | 0.58037414 | <.0001 |
| 10 | N5304 | 0.0467 | 14.6 | <.0001 | 0.162428 | <.0001 | 0.59073474 | <.0001 |
| 11 | N5514 | 0.0359 | 11.08 | <.0001 | 0.156597 | <.0001 | 0.59864879 | <.0001 |
| 12 | N4402 | 0.0278 | 8.48 | 0.0002 | 0.152251 | <.0001 | 0.60491478 | <.0001 |
| 13 | N2407 | 0.0275 | 8.37 | 0.0003 | 0.148069 | <.0001 | 0.61113503 | <.0001 |
| 14 | N4325 | 0.0288 | 8.78 | 0.0002 | 0.143803 | <.0001 | 0.61752281 | <.0001 |
| 15 | N5315 | 0.0391 | 12.03 | <.0001 | 0.13818 | <.0001 | 0.62507682 | <.0001 |
| 16 | N6315 | 0.0279 | 8.48 | 0.0002 | 0.134318 | <.0001 | 0.63017534 | <.0001 |
| 17 | N7304 | 0.0294 | 8.91 | 0.0002 | 0.130375 | <.0001 | 0.63564981 | <.0001 |
| 18 | N5302 | 0.0213 | 6.4 | 0.0018 | 0.127598 | <.0001 | 0.63903925 | <.0001 |
| 19 | N2502 | 0.0213 | 6.4 | 0.0018 | 0.124875 | <.0001 | 0.64251146 | <.0001 |
| 20 | N9311 | 0.0155 | 4.63 | 0.0102 | 0.122934 | <.0001 | 0.64541416 | <.0001 |
| 21 | N3417 | 0.0169 | 5.03 | 0.0068 | 0.120854 | <.0001 | 0.64865582 | <.0001 |
| 22 | N6214 | 0.0153 | 4.55 | 0.0109 | 0.119 | <.0001 | 0.651429 | <.0001 |
| 23 | N7616 | 0.0143 | 4.22 | 0.0151 | 0.117301 | <.0001 | 0.65419534 | <.0001 |
| 24 | N3307 | 0.014 | 4.13 | 0.0165 | 0.115658 | <.0001 | 0.65631134 | <.0001 |
| 25 | N7208 | 0.0105 | 3.09 | 0.0463 | 0.114441 | <.0001 | 0.65785918 | <.0001 |
| 26 | N6224 | 0.0117 | 3.42 | 0.0333 | 0.113107 | <.0001 | 0.65963436 | <.0001 |
| 27 | N2307 | 0.0106 | 3.1 | 0.0457 | 0.111907 | <.0001 | 0.66126359 | <.0001 |
| 28 | N6402 | 0.0092 | 2.69 | 0.0688 | 0.110875 | <.0001 | 0.6629722 | <.0001 |
| 29 | N5303 | 0.0078 | 2.26 | 0.1052 | 0.110013 | <.0001 | 0.66412334 | <.0001 |
| 30 | N3209 | 0.007 | 2.03 | 0.1324 | 0.109243 | <.0001 | 0.66519786 | <.0001 |
| 31 | N2401 | 0.0079 | 2.29 | 0.1019 | 0.108379 | <.0001 | 0.66631173 | <.0001 |
| 32 | N5123 | 0.0076 | 2.21 | 0.1106 | 0.107551 | <.0001 | 0.6673879 | <.0001 |
| 33 | N9312 | 0.0076 | 2.19 | 0.1125 | 0.106734 | <.0001 | 0.66858606 | <.0001 |
| 34 | N1406 | 0.0074 | 2.13 | 0.1203 | 0.105946 | <.0001 | 0.66960306 | <.0001 |
| 35 | N6519 | 0.007 | 2.01 | 0.1344 | 0.105815 | <.0001 | 0.66992024 | <.0001 |

When the concentration of drug treated patient serum samples were compared to the corresponding concentrations of these proteins in samples from drug naïve AD patient samples, a divergence in the serum concentrations of several proteins were noted.

Proteins involved in cellular degeneration (Group I), such as Apolipoproteins E4, E3, and A-IV, Transthyretin “dimer”, α-2 Macroglobulin, Glutathione-S-transferase Mu5-5, and Fidgitin I were less divergent from the age matched normal controls, while Apolipoprotein H and α-1 microglobulin, were divergent from the controls in the opposite direction with elevated levels vs. reduced levels in drug naïve patient samples.

Five of the haptoglobin proteins (i.e., Haptoglobin HP 2A; and Haptoglobin 1a, 1b, 1c, and 1d) involved in extra cellular and systemic oxidative stress response (Group II) had an elevated level vs. a reduced level in drug naïve patient samples; while the concentration of the Haptoglobin related protein was reduced. All of the Haptoglobin proteins were more divergent from the age matched normal controls than the corresponding proteins from drug naïve patient samples.

Proteins involved in humeral and cellular immune inflammatory responses (Group III) showed the most pronounced differences in concentrations between the drug treated AD patients and the drug naïve AD patients as well as control samples. For example, the DTAD patients exhibited markedly reduced levels of Inter-α-trypsin inhibitor heavy chain (H4) related 35 KD protein, Complement C3c1a, C3c1c, C3c2a, C3dg, and Factor Bb. In contrast, all of these proteins were substantially elevated in drug naïve patient samples. There were additional divergences in elevated levels of Immunoglobulins κ light chainiκc, and the Complement cytolysis inhibitor protein α-subunit vs. reduced levels in drug naïve patient samples, an elevated level of Complement Factor I vs. no change in level in drug naïve patient samples, and a less elevated level of Complement Factor H/Hs protein than in drug naïve patient samples.

Also divergent were three additional transport proteins (Group IV) and one of the two proteins of unknown function (X).

The marked differences between DNAD and DTAD were also seen in combination by multivariate linear discriminate analysis and receiver operator characteristics of the probabilities of diagnosis obtained (ROC), employing the combined concentrations of the 47 biomarkers to discriminate between prospectively collected and banked drug naïve AD patient samples (DNAD, n=44) vs. prospectively collected newly drawn AD patient samples from the clinical validation trial (DTAD, n=39), which showed a sensitivity of 95.5% for drug naïve AD and specificity of 96.1% for the group of clinical validation trial AD samples (DNAD vs. DTAD, FIG. 11).

Due to the markedly reduced divergence from normal of the prospectively collected newly drawn DTAD patient samples from the clinical validation trial from the age-matched normal controls, additional protein biomarkers were required for use in discriminant analysis to provide sufficient differential diagnostic discrimination of DTAD from age-matched normal and disease controls.

In addition, for drug treated disease controls 56 serum samples from Parkinson's disease patients, all of whom had been treated with dopamine agonists (therefore designated drug treated Parkinson's disease patients, DTPD), were investigated. Single biomarker concentration statistics of those patient samples combined with the existing 39 DTAD and 78 age-matched normal control samples from the two trials led to the identification of an additional ten serum protein biomarkers, which were then added to the multivariate discriminant analysis.

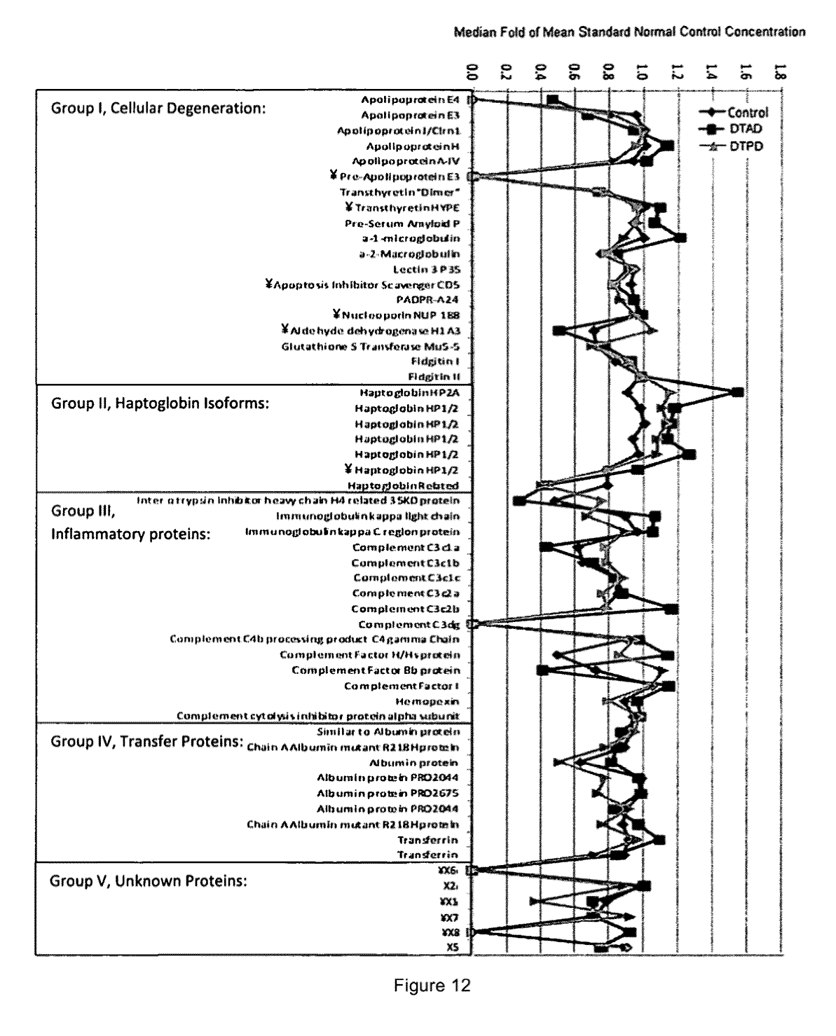

The profiles of the concentrations of the 57 serum proteins listed in Table 4 in prospectively collected newly drawn samples from the clinical validation trial from the DTAD, DTPD and age-matched normal controls were significantly different from one-another (FIG. 12).

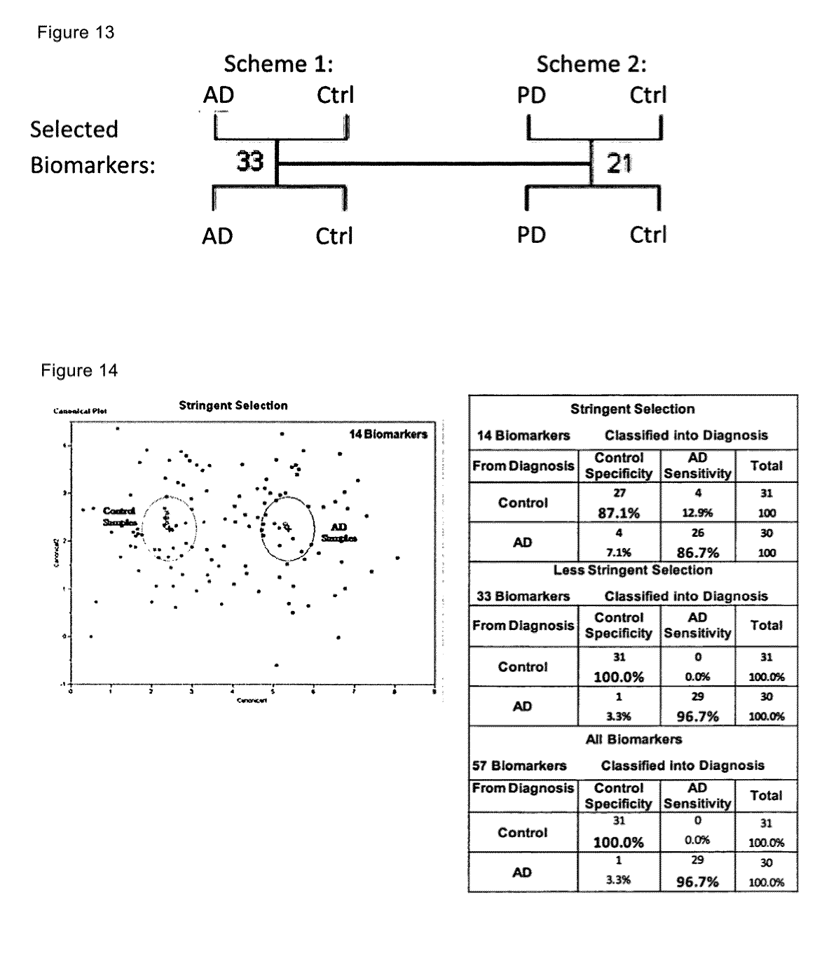

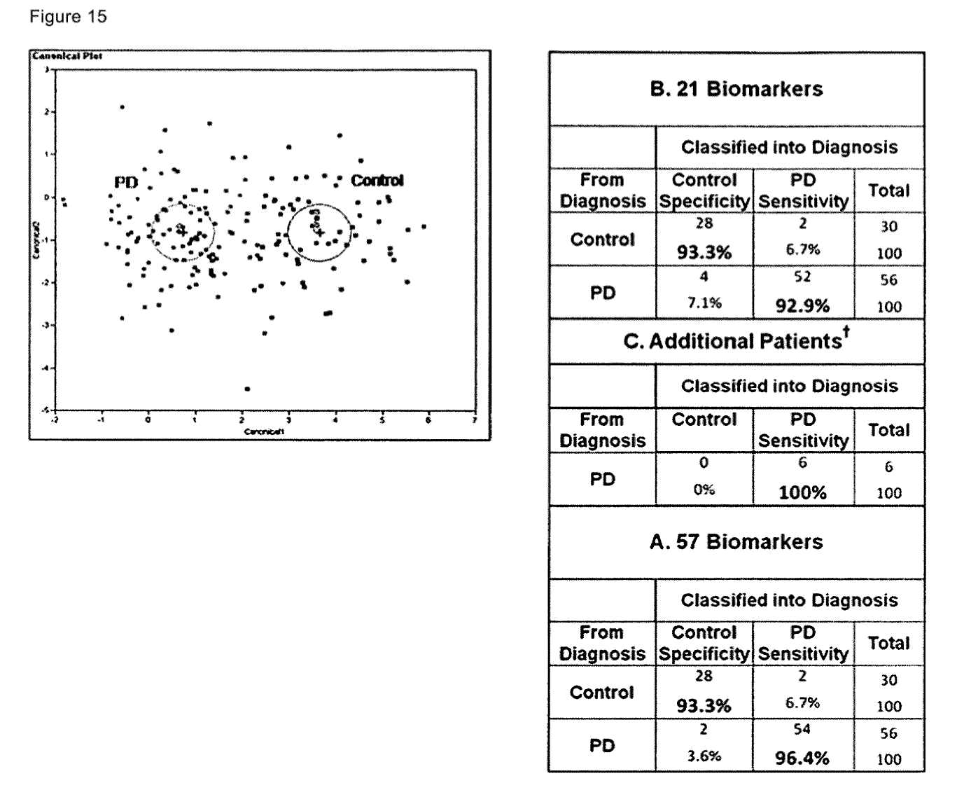

Stepwise linear discriminant analysis selected a combination of 33 of the 57 proteins as optimal to distinguish DTAD patients from controls (Sensitivity 96.7%, Specificity 100%) (see FIGS. 13-14, Table 6). A combination of 21 of the 57 proteins (including 12 of the 33 selected for DTAD vs. Control, see Table 7) were selected as optimal to distinguish DTPD patients from controls (Sensitivity 93.3%, Specificity 92.9%) (see FIGS. 13 and 15).

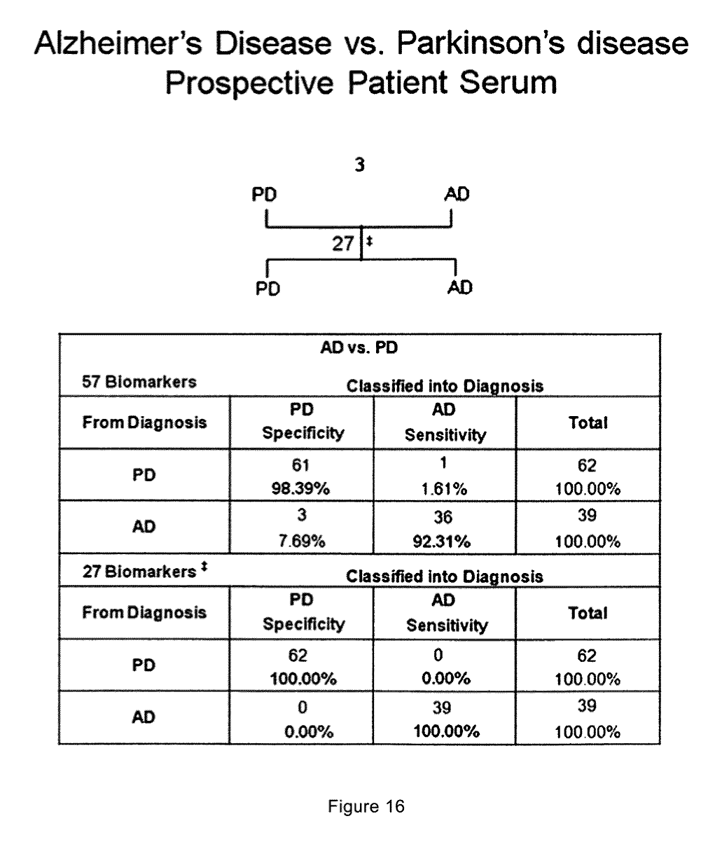

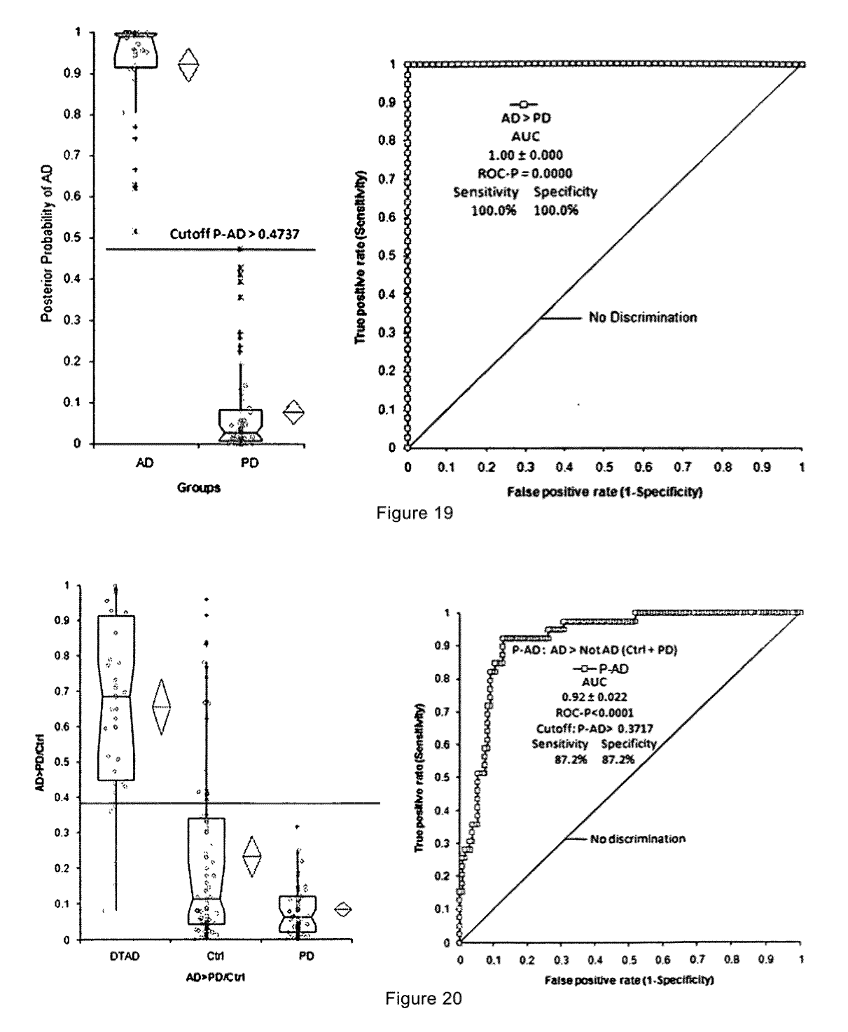

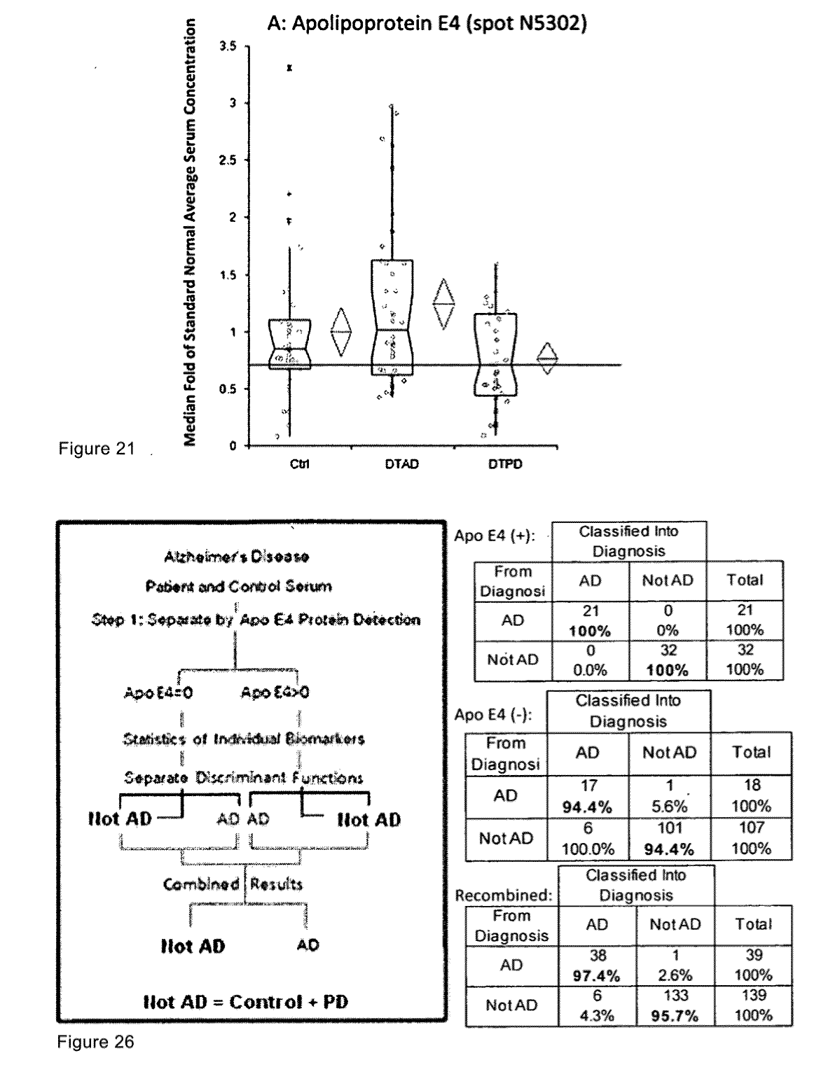

In addition, stepwise linear discriminant analysis selected a combination of 27 of the 57 proteins (including 13 of those selected for DTAD vs. Control, and 8 of the 21 selected for DTPD vs. Control, see Table 8) as optimal to distinguish DTAD patients from DTPD patients (Sensitivity 100%, Specificity 100%) (see FIGS. 16 and 19).

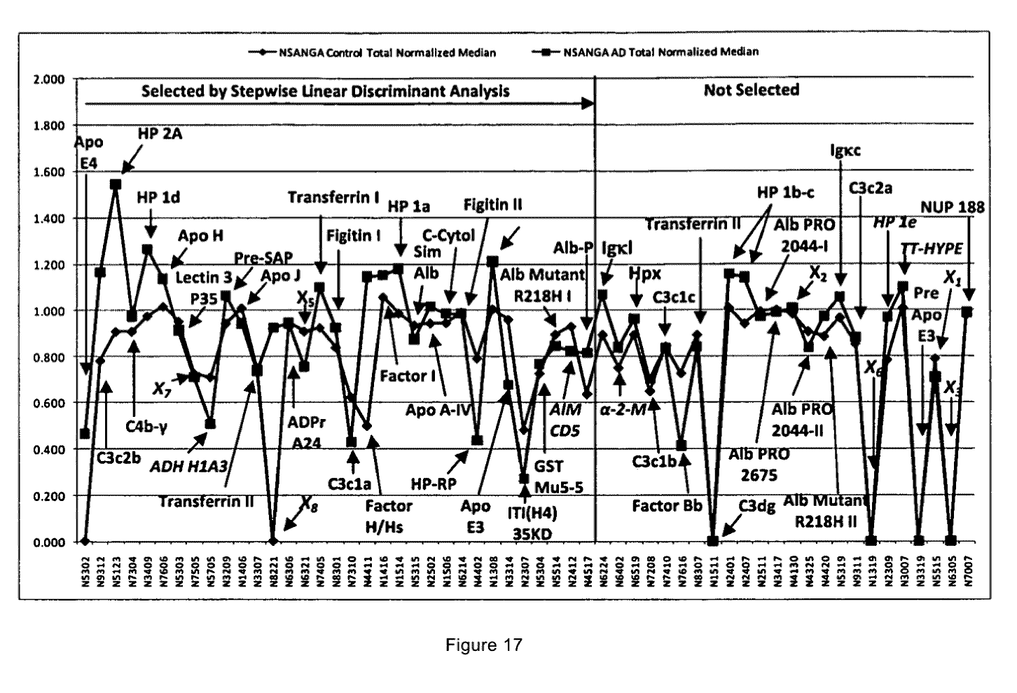

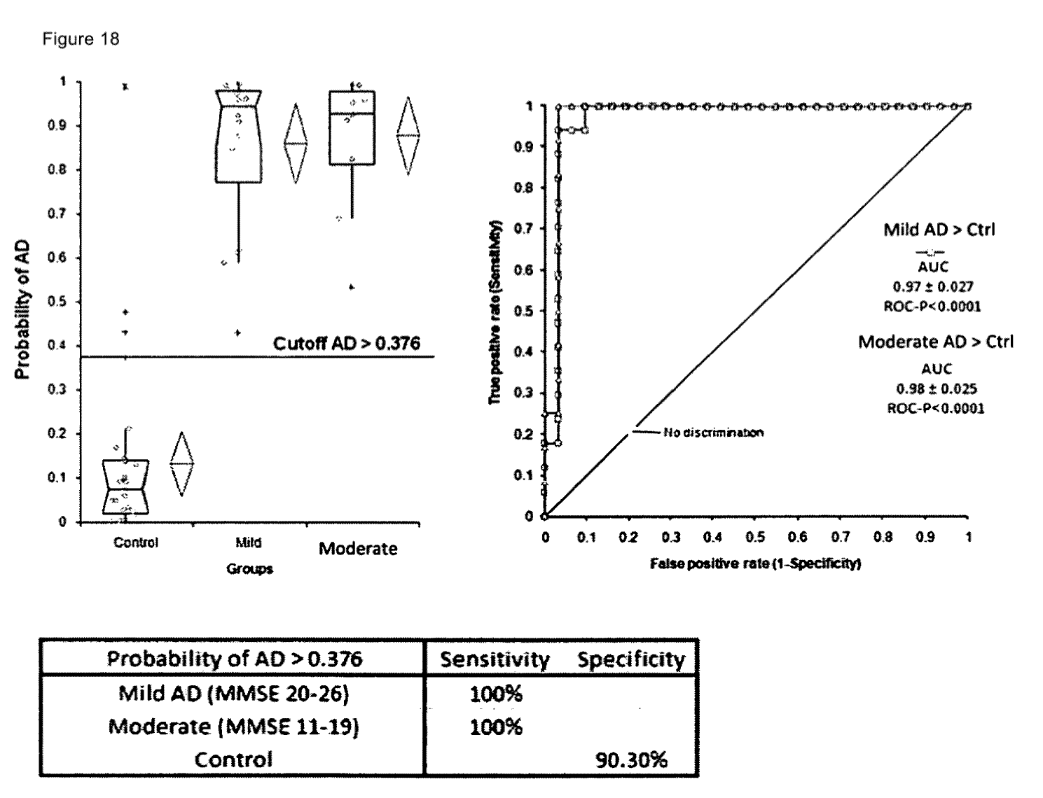

The sensitivity of distinguishing DTAD from age-matched normal controls using biomarkers selected by a stepwise linear discriminant analysis, over using biomarkers that were not selected, is shown in FIG. 17. Furthermore, the sensitivity of distinguishing DTAD from normal age-matched controls was essentially independent of the severity of the patients' symptoms (FIG. 18).

When multivariate discriminant analysis was performed by linear discriminant analysis (FIG. 20), employing the concentrations of 57 of these proteins, 3-way discrimination was obtained between 39 DTAD patients (Sensitivity=87.2%), 56 DTPD patients, and 78 age-matched normal controls (Combined specificity for Not AD=87.2%, Not AD=DTPD+age-matched normal controls).

Apo E4 Protein and AD Diagnosis

Apo E4 protein spot N5302 was the first protein selected by stepwise multivariate linear discriminant as providing the highest statistical contribution to AD diagnosis as compared to age-matched normal controls (Tables 6).

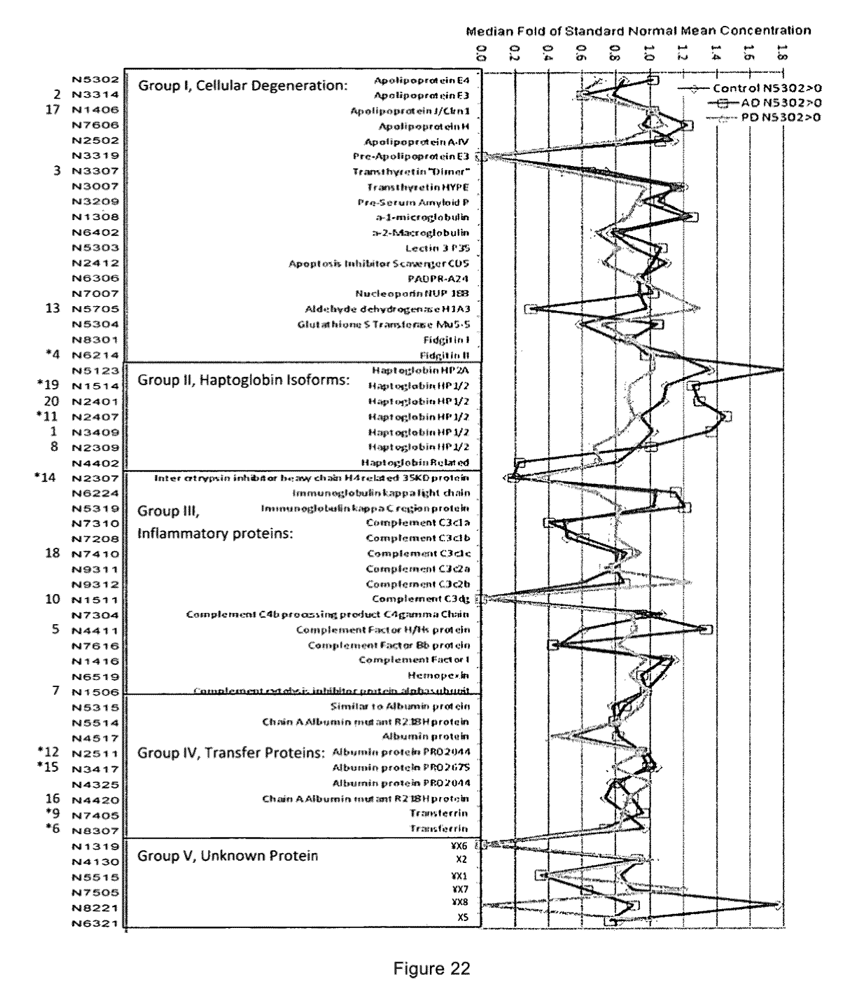

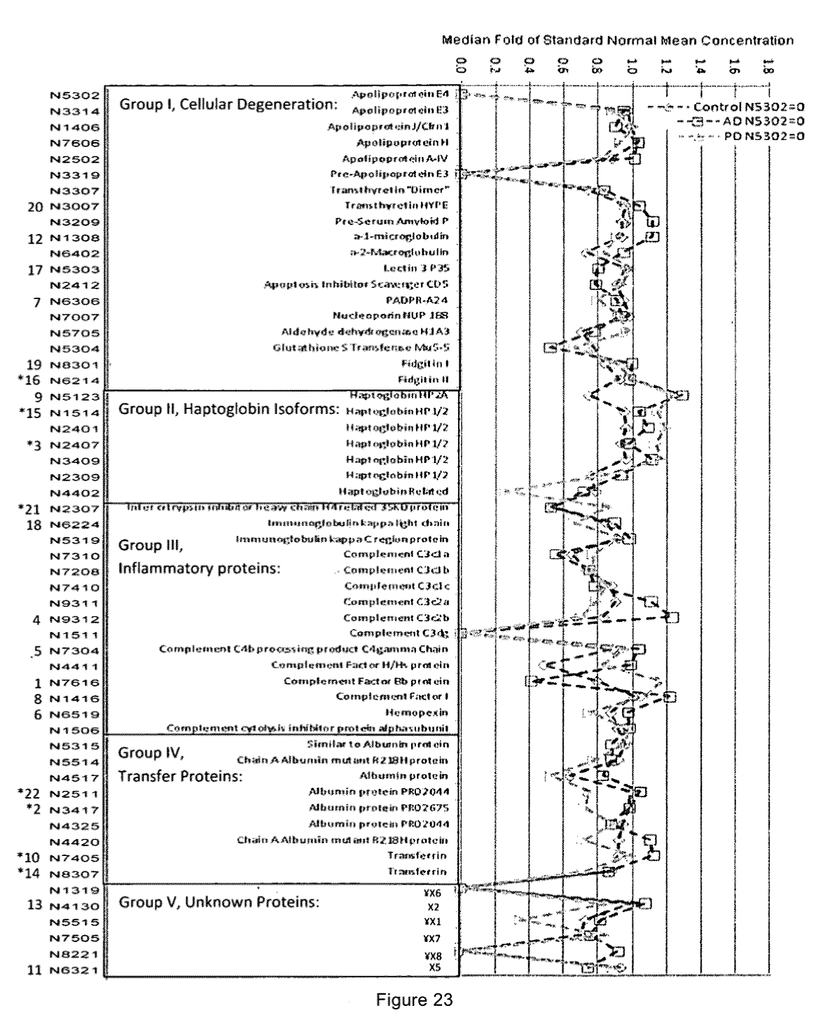

To investigate this further, the patients and controls were stratified into those patients with or without detectable amounts of the E4 protein in their sera. Upon stratification, significant differences in abnormal levels of the biomarkers were seen in the serum profiles of the Apo E4(+) DTAD and DTPD patients and the age-matched normal controls vs. Apo E4(−) patients and controls (FIGS. 22-23).

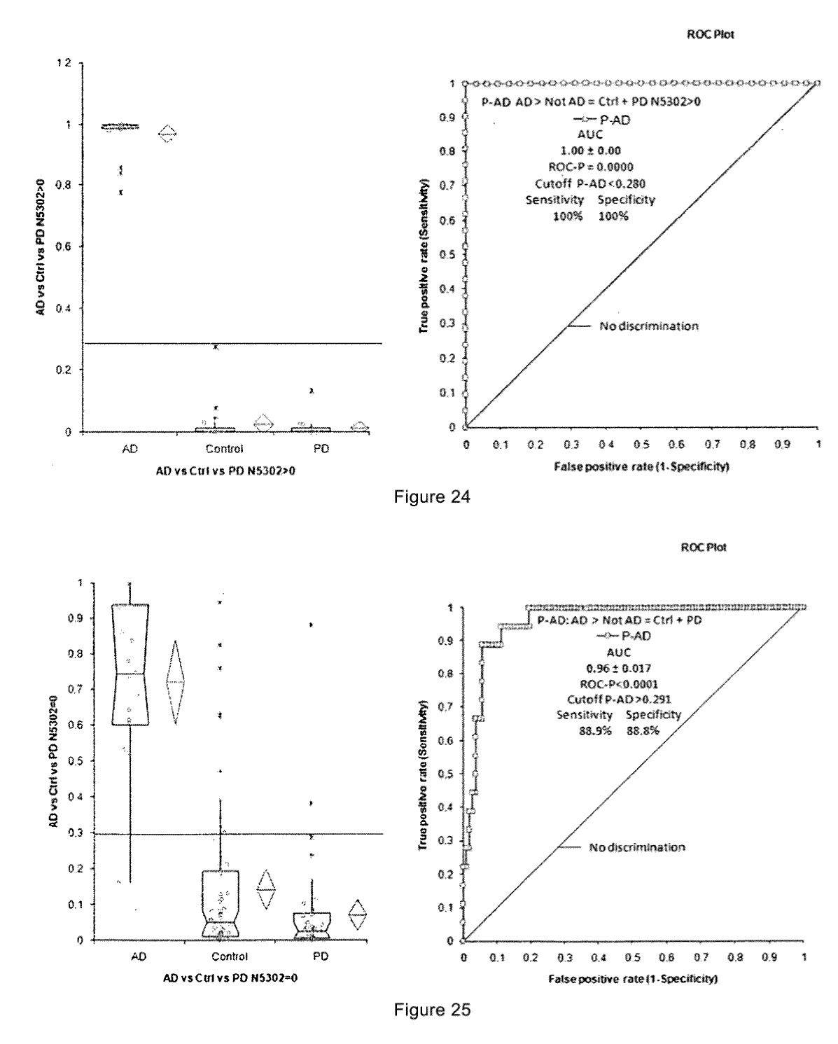

Furthermore, when multivariate linear discriminant analysis was employed on the stratified populations to discriminate DTAD vs. DTPD and age-matched normal controls (collectively Not-DTAD), those with detectable quantities of Apo E4 protein (with Apo E4(+) sera) were classified into DTAD and Not-DTAD with a sensitivity of 100% and a specificity of 100% (FIG. 24), whereas those without detectable quantities of Apo E4 protein (with Apo E4(−) sera) were classified into DTAD and Not DTAD with a sensitivity of 88.9% and a specificity of 88.8% (FIG. 25).

When the results of separate discriminant functions were tabulated by patient and combined, the overall sensitivity for DTAD was 97.4% and the overall specificity for Not DTAD was 95.7% (FIG. 26). In addition, stepwise linear discriminant analysis selected 20 proteins for Apo E4 (+) (Table 10), and 22 proteins for Apo E4 (−) (Table 11) as being optimally Complimentary for discriminating between DTAD and Not DTAD, with only 8 of the 47 proteins in common to both Apo E4(+) and Apo E4(−) linear discriminant functions (Table 9).

Thus, monitoring the concentrations of specified groups of serum proteins for the differential diagnosis of Alzheimer's disease patients, employing the concentrations of 57 serum proteins, and by using an initial separation of patients and normal and disease controls on the basis of detection or lack of detection in serum of Apolipoprotein E4 (the protein coded for by the high risk gene allele Apo E ε4 provided enhanced discrimination of drug treated patients with Alzheimer's disease (Sensitivity=97.4%) from those with Parkinson's disease and age-matched normal controls (Specificity=95.7%).

These results provide a basis of a blood test for differential diagnosis by a specialist, such as a neurologist or psychiatrist, of patients with suspected Alzheimer's disease who are already under treatment with anti dementia drugs, including donepezil, rivastigmine, memantine HCl, or the combination thereof. Such a test will be useful in the present clinical setting where by the time a neurologist sees a patient with suspected Alzheimer's disease, the patient is often already under treatment with one of these FDA approved anti-dementia drugs.

In addition, the significant differences in the serum protein profiles of Apo E4(+) and Apo E4(−) patients lends support to the concept that Apolipoprotein E4 confers differences in normal physiology, as well as in disease mechanisms, and in drug responses.

Implications for Differential Diagnosis

Embodiments of the present invention monitor the concentration (i.e., the up regulation or down regulation) of specified groups of serum proteins for the differential diagnosis of Alzheimer's disease patients.

### Embodiment 1

A screening/diagnostic assay for drug naïve AD patients is described that quantitates the serum concentration of 47 specific serum proteins in order to distinguish drug naïve Alzheimer's patients (Sensitivity=93.2%) from a group of drug naïve disease controls with AD-like Parkinson's disease, non-Alzheimer's and mixed dementias, and age-matched normal controls (Specificity=92.2%). Likewise, the assay can distinguish drug naïve AD patients from drug treated AD patients (Sensitivity=95.5%, Specificity=96.1%).

TABLE 6
Stepwise Multivariate Discriminant Selection Summary DTAD vs. Control

| | | | | | | | Average | | | |
|---|---|---|---|---|---|---|---|---|---|---|
| | | Partial | | | | | Squared | | | |
| | | R- | | | Wilks' | Pr < | Canonical | Pr > | | |
| Step | Biomarker | Square | F Value | Pr > F | Lambda | Lambda | Correlation | ASCC | Protein Spot | Protein ID |
| 1 | N5302 | 0.0793 | 10.34 | 0.0017 | 0.92065 | 0.0017 | 0.0793489 | 0.0017 | 1. N5302 Apo E4 | Apolipoprotein |
| | | | | | | | | | | E4 |
| 2 | N9312 | 0.0735 | 9.44 | 0.0026 | 0.853 | <.0001 | 0.1470038 | <.0001 | 2. N9312 C3c2b | Complement |
| | | | | | | | | | | C3c2b |
| 3 | N5123 | 0.0563 | 7.03 | 0.0091 | 0.80501 | <.0001 | 0.1949885 | <.0001 | 3. N5123 HP-2A | Haptoglobin |
| | | | | | | | | | | HP-2a Protein |
| 4 | N7304 | 0.058 | 7.21 | 0.0083 | 0.7583 | <.0001 | 0.2416984 | <.0001 | 4. N7304 C4bg | Complement |
| | | | | | | | | | | C4b Gamma |
| | | | | | | | | | | Chain Protein |
| 5 | N3409 | 0.0473 | 5.76 | 0.018 | 0.72246 | <.0001 | 0.2775439 | <.0001 | 5. N3409 HP-1d | Haptoglobin |
| | | | | | | | | | | HP-1d |
| 6 | N7606 | 0.0378 | 4.51 | 0.0358 | 0.69518 | <.0001 | 0.3048175 | <.0001 | 6. N7606 Apo H | Apolipoprotein H |
| 7 | N5303 | 0.0445 | 5.31 | 0.023 | 0.66423 | <.0001 | 0.3357739 | <.0001 | 7. N5303 L3 P35 | Lectin 3 P35 |
| 8 | N7505 | 0.0282 | 3.28 | 0.0729 | 0.64551 | <.0001 | 0.354494 | <.0001 | 8. N7505 X8 | X7 |
| 9 | N5705 | 0.0312 | 3.61 | 0.0601 | 0.62537 | <.0001 | 0.3746291 | <.0001 | 9. N5705 ADH1A3 | Aldehyde |
| | | | | | | | | | | Dehydrogenase |
| | | | | | | | | | | 1 A3 |
| 10 | N3209 | 0.0354 | 4.08 | 0.0459 | 0.60322 | <.0001 | 0.3967816 | <.0001 | 10. N3209 Pre-SAP | Pre-serum |
| | | | | | | | | | | Amyloid P |
| 11 | N1406 | 0.033 | 3.75 | 0.0554 | 0.58333 | <.0001 | 0.4166656 | <.0001 | 11. N1406 Clrn1 | Clusterin |
| | | | | | | | | | | Isoform 1 |
| 12 | N3307 | 0.027 | 3.02 | 0.085 | 0.5676 | <.0001 | 0.4324014 | <0001 | 12. N3307 TT“D” | Transthyretin |
| | | | | | | | | | | “Dimer” Protein |
| 13 | N8221 | 0.0203 | 2.23 | 0.1379 | 0.55609 | <.0001 | 0.4439074 | <.0001 | 13. N8221 X9 | X8 |
| 14 | N6306 | 0.0221 | 2.42 | 0.1229 | 0.54381 | <.0001 | 0.4561932 | <.0001 | 14. N6306 PDLaH | Acidic Histone |
| | | | | | | | | | | H2A Protein |
| | | | | | | | | | | (PD/LBD) |
| 15 | N6321 | 0.0193 | 2.09 | 0.1515 | 0.53331 | <.0001 | 0.4666922 | <.0001 | 15. N6321 X5 | X5 |
| 16 | N7405 | 0.019 | 2.03 | 0.1567 | 0.52317 | <.0001 | 0.4768306 | <.0001 | 16. N7405 TTR1 | Transferrin |
| | | | | | | | | | | Protein I |
| 17 | N8301 | 0.05 | 5.48 | 0.0212 | 0.497 | <.0001 | 0.5029991 | <.0001 | 17. N8301 Fidgitin I | Fidgitin Protein I |
| 18 | N7310 | 0.026 | 2.75 | 0.1003 | 0.48407 | <.0001 | 0.5159253 | <.0001 | 18. N7310 C3cla | Complement |
| | | | | | | | | | | C3c1a |
| 19 | N4411 | 0.0181 | 1.88 | 0.1737 | 0.47533 | <.0001 | 0.5246731 | <.0001 | 19. N4411 Factor | Complement |
| | | | | | | | | | H/Hs | Factor H/Hs |
| | | | | | | | | | | Protein |
| 20 | N1416 | 0.0209 | 2.15 | 0.1452 | 0.4654 | <.0001 | 0.5346019 | <.0001 | 20. N1416 Factor I | Complement |
| | | | | | | | | | | Factor 1 Protein |
| 21 | N1514 | 0.0382 | 3.97 | 0.049 | 0.44763 | <.0001 | 0.552375 | <.0001 | 21. N1514 HP-1a | Haptoglobin |
| | | | | | | | | | | HP-1a Protein |
| 22 | N5315 | 0.0207 | 2.1 | 0.1508 | 0.43834 | <.0001 | 0.5616581 | <.0001 | 22. N5315 Sim-Alb | Similar to |
| | | | | | | | | | | Albumin |
| 23 | N2502 | 0.0284 | 2.87 | 0.0937 | 0.42589 | <.0001 | 0.5741108 | <.0001 | 23. N2502 Apo A-IV | Apolipoprotein |
| | | | | | | | | | | A-IV Protein |
| 24 | N1506 | 0.0193 | 1.91 | 0.1705 | 0.41768 | <.0001 | 0.5823214 | <.0001 | 24. N1405 Cyp | Complement |
| | | | | | | | | | | Cytolysis |
| | | | | | | | | | | Protein |
| 25 | N6214 | 0.0232 | 2.28 | 0.1345 | 0.408 | <.0001 | 0.5920016 | <.0001 | 25. N6214 Fidgitin II | Fidgitin Protein |
| | | | | | | | | | | II |
| 26 | N4402 | 0.0197 | 1.91 | 0.1704 | 0.39996 | <.0001 | 0.6000355 | <.0001 | 26. N4402 HP-RP | Haptoglobin |
| | | | | | | | | | | Related Protein |
| 27 | N1308 | 0.0195 | 1.87 | 0.1748 | 0.39217 | <.0001 | 0.6078345 | <.0001 | 27. N1308a-1mg | Alpha-1- |
| | | | | | | | | | | microglobulin |
| 28 | N3314 | 0.0254 | 2.42 | 0.1233 | 0.38222 | <.0001 | 0.6177762 | <.0001 | 28. N3314 Apo E3 | Apolipoprotein |
| | | | | | | | | | | E3 Protein |
| 29 | N2307 | 0.0233 | 2.19 | 0.1421 | 0.37333 | <.0001 | 0.6266736 | <.0001 | 29. N2307 ITI (H4) | Int-a-tryp Inh |
| | | | | | | | | | RP 35 KD | HC (H4) Rel |
| | | | | | | | | | | 35 KD Protein |
| 30 | N5304 | 0.0213 | 1.98 | 0.163 | 0.36538 | <.0001 | 0.6346166 | <.0001 | 30. N5304 | Glutathione- |
| | | | | | | | | | GSTMu5-5 | s-transferase |
| | | | | | | | | | | Mu 5-5 |
| 31 | N5514 | 0.0206 | 1.89 | 0.1723 | 0.35786 | <.0001 | 0.6421422 | <.0001 | 31. N5514 Alb | Chain A |
| | | | | | | | | | Mutant R218H-I | Albumin Mutant |
| | | | | | | | | | | R218H Protein |
| 32 | N2412 | 0.0187 | 1.69 | 0.1965 | 0.35117 | <.0001 | 0.6488252 | <.0001 | 32. N2412 AIM CD-5 | Apoptiosis |
| | | | | | | | | | | Inhibitor |
| | | | | | | | | | | Scavenger |
| | | | | | | | | | | Receptor |
| 33 | N4517 | 0.0192 | 1.72 | 0.1929 | 0.34444 | <.0001 | 0.6555642 | <.0001 | 33. N4517 AlbP | Albumin Protein |

TABLE 7
Stepwise Multivariate Discriminant Selection Summary DTPD vs. Control

| | | | | | | | Average | | | |
|---|---|---|---|---|---|---|---|---|---|---|
| | | | | | | | Squared | | | |
| | | Partial | | | Wilks' | Pr < | Canonical | | | |
| Step | Label | R-Square | F Value | Pr > F | Lambda | Lambda | Correlation | Pr > ASCC | Biomarker | Protein ID |
| 1 | N5514 | 0.1019 | 26 | <.0001 | 0.8981 | <.0001 | 0.1019 | <.0001 | N5514 | Chain A Albumin |
| | | | | | | | | | | Mutant R218H |
| | | | | | | | | | | Protein |
| 2 | N5123 | 0.0594 | 14.39 | 0.0002 | 0.8447 | <.0001 | 0.1553 | <.0001 | N5123 | Haptoglobin HP-2a |
| | | | | | | | | | | Protein |
| 3 | N5515 | 0.0592 | 14.3 | 0.0002 | 0.7947 | <.0001 | 0.2053 | <.0001 | N5515 | X1 Protein |
| 4 | N1416 | 0.0538 | 12.86 | 0.0004 | 0.7519 | <.0001 | 0.2481 | <.0001 | N1416 | Complement Factor I |
| | | | | | | | | | | Protein |
| 5 | N3314 | 0.0432 | 10.16 | 0.0016 | 0.7194 | <.0001 | 0.2806 | <.0001 | N3314 | Apolipoprotein E3 |
| | | | | | | | | | | Protein |
| 6 | N3307 | 0.0435 | 10.18 | 0.0016 | 0.6881 | <.0001 | 0.3119 | <.0001 | N3307 | Transthyretin |
| | | | | | | | | | | “Dimer” Protein |
| 7 | N7007 | 0.0444 | 10.37 | 0.0015 | 0.6576 | <.0001 | 0.3424 | <.0001 | N7007 | Nucleoporin NUP188 |
| | | | | | | | | | | Protein |
| 8 | N2407 | 0.0382 | 8.82 | 0.0033 | 0.6324 | <.0001 | 0.3676 | <.0001 | N2407 | Haptoglobin HP-1 |
| | | | | | | | | | | Protein |
| 9 | N2511 | 0.052 | 12.13 | 0.0006 | 0.5995 | <.0001 | 0.4005 | <.0001 | N2511 | Albumin Protein |
| | | | | | | | | | | PRO2044 |
| 10 | N6306 | 0.0316 | 7.18 | 0.0079 | 0.5806 | <.0001 | 0.4194 | <.0001 | N6306 | PDLaH acidic H2A |
| | | | | | | | | | | (ADPR/ub/A24) |
| | | | | | | | | | | Protein |
| 11 | N2502 | 0.0261 | 5.87 | 0.0163 | 0.5654 | <.0001 | 0.4346 | <.0001 | N2502 | Apolipoprotein A-IV |
| | | | | | | | | | | Protein |
| 12 | N3007 | 0.0291 | 6.54 | 0.0113 | 0.549 | <.0001 | 0.451 | <.0001 | N3007 | Transthyretin HYPE |
| | | | | | | | | | | Protein |
| 13 | N7304 | 0.0248 | 5.52 | 0.0196 | 0.5353 | <.0001 | 0.4647 | <.0001 | N7304 | Complement C4b |
| | | | | | | | | | | Gamma Chain Protein |
| 14 | N4420 | 0.0233 | 5.14 | 0.0243 | 0.5229 | <.0001 | 0.4771 | <.0001 | N4420 | Chain A Albumin |
| | | | | | | | | | | mutant R218H |
| | | | | | | | | | | Protein |
| 15 | N8301 | 0.0179 | 3.93 | 0.0488 | 0.5135 | <.0001 | 0.4865 | <.0001 | N8301 | Fidgitin Protein 1 |
| 16 | N6224 | 0.0159 | 3.45 | 0.0646 | 0.5054 | <.0001 | 0.4946 | <.0001 | N6224 | Immunoglobulin |
| | | | | | | | | | | Kappa Light Chain |
| | | | | | | | | | | Protein |
| 17 | N4411 | 0.0173 | 3.75 | 0.0543 | 0.4966 | <.0001 | 0.5034 | <.0001 | N4411 | Complement Factor |
| | | | | | | | | | | H/Hs Protein |
| 18 | N6214 | 0.0147 | 3.17 | 0.1764 | 0.4893 | <.0001 | 0.5107 | <.0001 | N6214 | Fidgitin Protein II |
| 19 | N3417 | 0.0115 | 2.46 | 0.1186 | 0.4837 | <.0001 | 0.5163 | <.0001 | N3417 | Albumin Protein |
| | | | | | | | | | | PRO2675 Protein |
| 20 | N4130 | 0.0149 | 3.17 | 0.0766 | 0.4765 | <.0001 | 0.5235 | <.0001 | N4130 | X2 Protein |
| 21 | N4402 | 0.0134 | 2.83 | 0.0937 | 0.4701 | <.0001 | 0.5299 | <.0001 | N4402 | Haptoglobin Related |
| | | | | | | | | | | Protein |

TABLE 8
Stepwise Multivariate Discriminant Selection Summary DTAD vs. DTPD

| | | | | | | | Average | | | |
|---|---|---|---|---|---|---|---|---|---|---|
| | | Partial | | | | | Squared | | | |
| | | R- | F | | Wilks' | Pr < | Canonical | Pr > | | |
| Step | Biomarker | Square | Value | Pr > F | Lambda | Lambda | Correlation | ASCC | Protein Spot | Protein ID |
| 1 | N7616 | 0.1846 | 54.56 | <.0001 | 0.81539065 | <.0001 | 0.18460935 | <.0001 | 1. N7616 Factor | Complement Factor |
| | | | | | | | | | Bb | Bb |
| 2 | N6224 | 0.1393 | 38.83 | <.0001 | 0.70184299 | <.0001 | 0.29815701 | <.0001 | 2. N6224 lgkl | Immunoglobulin |
| | | | | | | | | | | Kappa Light Chain |
| | | | | | | | | | | Protein |
| 3 | N1308 | 0.1010 | 26.86 | <.0001 | 0.63093381 | <.0001 | 0.36906619 | <.0001 | 3. N1308 a-1 mg | Alpha-1- |
| | | | | | | | | | | microglobulin |
| 4 | N5302 | 0.0724 | 18.57 | <.0001 | 0.58526715 | <.0001 | 0.41473285 | <.0001 | 4. N5302 Apo | Apolipoprotein E4 |
| | | | | | | | | | E4 | |
| 5 | N6519 | 0.0508 | 12.69 | 0.0004 | 0.55553212 | <.0001 | 0.44446788 | <.0001 | 5. N6519 HPX | Hemopexin |
| 6 | N5705 | 0.0421 | 10.38 | 0.0015 | 0.53211910 | <.0001 | 0.46788090 | <.0001 | 6. N5705 | Aldehyde |
| | | | | | | | | | ADH1A3 | Dehydrogenase 1 A3 |
| 7 | N3417 | 0.0432 | 10.62 | 0.0013 | 0.50911077 | <.0001 | 0.49088923 | <.0001 | 7. N3417 Alb | Albumin Protein |
| | | | | | | | | | PRO2675 | PRO2675 Protein |
| 8 | N4325 | 0.0647 | 16.19 | <.0001 | 0.47615657 | <.0001 | 0.52384343 | <.0001 | 8. N4325 | Albumin PR02044 II |
| | | | | | | | | | PR02044 II | |
| 9 | N2307 | 0.0385 | 9.33 | 0.0025 | 0.45782793 | <.0001 | 0.54217207 | <.0001 | 9. N307 ITI | Int-a-tryp Inh HC |
| | | | | | | | | | (H4) RP | (H4) Rel 35 KD |
| | | | | | | | | | 35 KD | Protein |
| 10 | N4130 | 0.0329 | 7.90 | 0.0054 | 0.44275296 | <.0001 | 0.55724704 | <.0001 | 10. N4130 X2 | X2 Protein |
| 11 | N4420 | 0.0357 | 8.56 | 0.0038 | 0.42692668 | <.0001 | 0.57307332 | <.0001 | 11. N4420 Alb | Alb Mutant |
| | | | | | | | | | R218H-II | R218H-II |
| 12 | N4517 | 0.0356 | 8.49 | 0.0039 | 0.41173690 | <.0001 | 0.58826310 | <.0001 | 12. N4517 AlbP | Albumin Protein |
| 13 | N1406 | 0.0232 | 5.44 | 0.0206 | 0.40218648 | <.0001 | 0.59781352 | <.0001 | 13. N1406 Clrn1 | Clusterin Isoform 1 |
| 14 | N1416 | 0.0387 | 9.17 | 0.0027 | 0.38664073 | <.0001 | 0.61335927 | <.0001 | 14. N1416 | Complement |
| | | | | | | | | | Factor I | Factor I Protein |
| 15 | N7304 | 0.0219 | 5.09 | 0.0250 | 0.37816446 | <.0001 | 0.62183554 | <.0001 | 15. N7304C4bg | Complement C4b |
| | | | | | | | | | | Gamma Chain |
| | | | | | | | | | | Protein |
| 16 | N7505 | 0.0201 | 4.64 | 0.0323 | 0.37055440 | <.0001 | 0.62944560 | <.0001 | 16. N7505 X7 | X7 Protein |
| 17 | N5319 | 0.0188 | 4.31 | 0.0389 | 0.36358267 | <.0001 | 0.63641733 | <.0001 | 17. N5319 lkc | Immunoglobulin |
| | | | | | | | | | | Kappa C Region |
| | | | | | | | | | | Protein |
| 18 | N1511 | 0.0156 | 3.55 | 0.0607 | 0.35790265 | <.0001 | 0.64209735 | <.0001 | 18. N1511 C3dg | Complement C3dg |
| 19 | N6321 | 0.0171 | 3.89 | 0.0498 | 0.35176474 | <.0001 | 0.64823526 | <.0001 | 19. N6321X5 | X5 Protein |
| 20 | N7405 | 0.0242 | 5.50 | 0.0199 | 0.34326708 | <.0001 | 0.65673292 | <.0001 | 20. N7405 TTR1 | Transferrin Protein I |
| 21 | N1514 | 0.0185 | 4.18 | 0.0422 | 0.33690041 | <.0001 | 0.66309959 | <.0001 | 21. N1514 HP-1a | Haptoglobin HP-1a |
| | | | | | | | | | | Protein |
| 22 | N3319 | 0.0171 | 3.83 | 0.0517 | 0.33114133 | <.0001 | 0.66885867 | <.0001 | 22. N3319 | Pro-Apolipoprotein |
| | | | | | | | | | Pre-Apo E3 | E3 |
| 23 | N7007 | 0.0116 | 2.56 | 0.1108 | 0.32731016 | <.0001 | 0.67268984 | <.0001 | 23. N7007 NUP | Nucleoporin NUP |
| | | | | | | | | | 188 | 188 Protein |
| 24 | N7208 | 0.0095 | 2.09 | 0.1495 | 0.32419852 | <.0001 | 0.67580148 | <.0001 | 24. N7208 C3c1b | Complement C3c1b |
| 25 | N7410 | 0.0118 | 2.59 | 0.1087 | 0.32036834 | <.0001 | 0.67963166 | <.0001 | 25. N7410 C3c1c | Complement C3c1c |
| 26 | N5515 | 0.0164 | 3.60 | 0.0592 | 0.31511839 | <.0001 | 0.68488161 | <.0001 | 26. N5515 X1 | X1 Protein |
| 27 | N9312 | 0.0125 | 2.72 | 0.1004 | 0.31117786 | <.0001 | 0.68882214 | <.0001 | 27. N9312 C3c2b | Complement C3c2b |

TABLE 9
Biomarkers Selected by Stepwise Discriminant analysis for
DTAD vs. Control, DTPD vs. Control and DTAD vs DTPD^{¥}

| | | | | |
|---|---|---|---|---|
| 1. | Apolipoprotein E4 | AD/Ctrl | | AD/PD (4) |
| 2. | Complement C3c2b | AD/Ctrl | | AD/PD (27) |
| 3. | Haptoglobin HP-2a Protein | AD/Ctrl | PD/Ctrl (2) | |
| 4. | Complement C4b Gamma Chain Protein | AD/Ctrl | PD/Ctrl (13) | AD/PD (15) |
| 5. | Haptoglobin HP-1d | AD/Ctrl | | |
| 6. | Apolipoprotein H | AD/Ctrl | | |
| 7. | Lectin 3 P35 | AD/Ctrl | | |
| 8. | X7 | AD/Ctrl | | AD/PD (16) |
| 9. | Aldehyde Dehydrogenase 1 A3 | AD/Ctrl | | AD/PD (6) |
| 10. | Pre-serum Amyloid P | AD/Ctrl | | |
| 11. | Clusterin Isoform 1 | AD/Ctrl | | AD/PD (13) |
| 12. | Transthyretin “Dimer” Protein | AD/Ctrl | PD/Ctrl (6) | |
| 13. | X8 | AD/Ctrl | | |
| 14. | Acidic Histone H2A Protein (PD/LBD) | AD/Ctrl | PD/Ctrl (10) | |
| 15. | X5 | AD/Ctrl | | AD/PD (19) |
| 16. | Transferrin Protein I | AD/Ctrl | | AD/PD (20) |
| 17. | Fidgitin Protein I | AD/Ctrl | PD/Ctrl (15) | |
| 18. | Complement C3c1a | AD/Ctrl | | |
| 19. | Complement Factor H/Hs Protein | AD/Ctrl | PD/Ctrl (17) | |
| 20. | Complement Factor I Protein | AD/Ctrl | PD/Ctrl (4) | AD/PD (14) |
| 21. | Haptoglobin HP-1a Protein | AD/Ctrl | | AD/PD (21) |
| 22. | Similar to Albumin | AD/Ctrl | | |
| 23. | Apolipoprotein A-IV Protein | AD/Ctrl | PD/Ctrl (11) | |
| 24. | Complement Cytolysis Protein | AD/Ctrl | | |
| 25. | Fidgitin Protein II | AD/Ctrl | PD/Ctrl (18) | |
| 26. | Haptoglobin Related Protein | AD/Ctrl | PD/Ctrl (21) | |
| 27. | Alpha-1-microglobulin | AD/Ctrl | | AD/PD (3) |
| 28. | Apolipoprotein E3 Protein | AD/Ctrl | PD/Ctrl (5) | |
| 29. | Int-α-tryp Inh HC (H4) Rel 35 KD Protein | AD/Ctrl | | AD/PD (9) |
| 30. | Glutathione -s-transferase Mu 5-5 | AD/Ctrl | | |
| 31. | Chain A Albumin Mutant R218H Protein | AD/Ctrl | PD/Ctrl (1) | |
| 32. | Apoptosis Inhibitor Scavenger Receptor | AD/Ctrl | | |
| 33. | Albumin Protein | AD/Ctrl | | AD/PD (12) |
| 34. | X1 Protein | | PD/Ctrl (3) | AD/PD (26)) |
| 35. | Nucleoporin NUP 188 Protein | | PD/Ctrl (7) | AD/PD (23) |
| 36. | Haptoglobin HP-1 Protein | | PD/Ctrl (8) | |
| 37. | Albumin Protein PRO2044 | | PD/Ctrl (9) | |
| 38. | Transthyretin HYPE Protein | | PD/Ctrl (12) | |
| 39. | Chain A Albumin mutant R218H Protein | | PD/Ctrl(14) | AD/PD (11) |
| 40. | Immunoglobulin Kappa Light Chain Protein | | PD/Ctrl (16) | AD/PD (2) |
| 41. | Albumin Protein PRO2675 Protein | | PD/Ctrl (19) | AD/PD (7) |
| 42. | X2 Protein | | PD/Ct6rl (20) | AD/PD (10) |
| 43. | Complement C3dg | | | AD/PD (18) |
| 44. | Pre-Apolipoprotein E3 | | | AD/PD (22) |
| 45. | Albumin PR02044 II | | | AD/PD (8) |
| 46. | Immunoglobulin Kappa C Region Protein | | | AD/PD (17) |
| 47. | Hemopexin | | | AD/PD (5) |
| 48. | Complement C3c1b | | | AD/PD (24) |
| 49. | Complement C3c1c | | | AD/PD (25) |
| 50. | Complement C3c1c | | | AD/PD (25) |
| 51. | Alpha-2-Macroglobulin | | | |
| 52. | Haptoglobin HP-1b Protein | | | |
| 53. | Haptoglobin HP-1e Protein | | | |
| 54. | Complement C3c2a Protein | | | |
| 55. | Transferrin Protein II | | | |
| 56. | X6 Protein | | | |
| 57. | X3 Protein | | | |
| 58. | X4 Protein | | | |

TABLE 10
Apo E4 (+)
AD vs PD vs Control 5302 = 0
The STEPDISC Procedure
Stepwise Selection Summary

| | | | | | | | Average | |
|---|---|---|---|---|---|---|---|---|
| | | | | | | | Squared | |
| | | Partial R- | | | Wilks' | Pr < | Canonical | Pr > |
| Step | Label | Square | F Value | Pr > F | Lambda | Lambda | Correlation | ASCC |
| 1 | N7616 | 0.107 | 17.91 | <.0001 | 0.89302 | <.0001 | 0.05348941 | <.0001 |
| 2 | N3417 | 0.0914 | 14.99 | <.0001 | 0.81138 | <.0001 | 0.09567486 | <.0001 |
| 3 | N2407 | 0.0743 | 11.91 | <.0001 | 0.75113 | <.0001 | 0.12731168 | <.0001 |
| 4 | N9312 | 0.0652 | 10.32 | <.0001 | 0.70215 | <.0001 | 0.15583828 | <.0001 |
| 5 | N7304 | 0.0507 | 7.87 | 0.0005 | 0.66657 | <.0001 | 0.17805636 | <.0001 |
| 6 | N6519 | 0.0514 | 7.97 | 0.0004 | 0.63227 | <.0001 | 0.19717401 | <.0001 |
| 7 | N6306 | 0.0389 | 5.94 | 0.003 | 0.60765 | <.0001 | 0.21285299 | <.0001 |
| 8 | N1416 | 0.0382 | 5.8 | 0.0034 | 0.58443 | <.0001 | 0.22897042 | <.0001 |
| 9 | N5123 | 0.0487 | 7.45 | 0.0007 | 0.55595 | <.0001 | 0.24939871 | <.0001 |
| 10 | N7405 | 0.0334 | 5.01 | 0.0072 | 0.53737 | <.0001 | 0.26336658 | <.0001 |
| 11 | N6321 | 0.037 | 5.55 | 0.0043 | 0.51748 | <.0001 | 0.27791801 | <.0001 |
| 12 | N1308 | 0.0285 | 4.23 | 0.0155 | 0.50273 | <.0001 | 0.28751745 | <.0001 |
| 13 | N4130 | 0.0254 | 3.74 | 0.0249 | 0.48996 | <.0001 | 0.29711088 | <.0001 |
| 14 | N8307 | 0.0232 | 3.4 | 0.0348 | 0.47859 | <.0001 | 0.30586415 | <.0001 |
| 15 | N1514 | 0.0201 | 2.92 | 0.0555 | 0.46898 | <.0001 | 0.31338762 | <.0001 |
| 16 | N6214 | 0.0231 | 3.36 | 0.0362 | 0.45814 | <.0001 | 0.32163852 | <.0001 |
| 17 | N5303 | 0.0242 | 3.51 | 0.0313 | 0.44706 | <.0001 | 0.33036203 | <.0001 |
| 18 | N6224 | 0.0234 | 3.38 | 0.0355 | 0.4366 | <.0001 | 0.33818938 | <.0001 |
| 19 | N8301 | 0.0234 | 3.37 | 0.0357 | 0.42636 | <.0001 | 0.34605255 | <.0001 |
| 20 | N3007 | 0.0191 | 2.72 | 0.0676 | 0.41824 | <.0001 | 0.35211982 | <.0001 |
| 21 | N2307 | 0.0181 | 2.56 | 0.0788 | 0.41069 | <.0001 | 0.35762711 | <.0001 |
| 22 | N2511 | 0.0181 | 2.56 | 0.0794 | 0.40327 | <.0001 | 0.36312094 | <.0001 |

TABLE 11
Biomarkers Selected by Stepwise Discriminant Analysis for
DTAD vs. Control, DTPD vs. Control, and DTAD vs. DTPD^{¥}

| | Protein ID | Selected by Stepwise Discriminant for: | | |
|---|---|---|---|---|
| 1. | Apolipoprotein E4 | AD/Ctrl | | AD/PD (4) |
| 2. | Complement C3c2b | AD/Ctrl | | AD/PD (27) |
| 3. | Haptoglobin HP-2a Protein | AD/Ctrl | PD/Ctrl (2) | |
| 4. | Complement C4b Gamma Chain Protein | AD/Ctrl | PD/Ctrl(13) | AD/PD (15) |
| 5. | Haptoglobin HP-1d | AD/Ctrl | | |
| 6. | Apolipoprotein H | AD/Ctrl | | |
| 7. | Lectin 3 P35 | AD/Ctrl | | |
| 8. | X7 | AD/Ctrl | | AD/PD (16) |
| 9. | Aldehyde Dehydrogenase 1 A3 | AD/Ctrl | | AD/PD (6) |
| 10. | Pre-serum Amyloid P | AD/Ctrl | | |
| 11. | Clusterin Isoform 1 | AD/Ctrl | | AD/PD (13) |
| 12. | Transthyretin “Dimer” Protein | AD/Ctrl | PD/Ctrl (6) | |
| 13. | X8 | AD/Ctrl | | |
| 14. | Acidic Histone H2A Protein (PD/LBD) | AD/Ctrl | PD/Ctrl (10) | |
| 15. | X5 | AD/Ctrl | | AD/PD (19) |
| 16. | Transferrin Protein I | AD/Ctrl | | AD/PD (20) |
| 17. | Fidgitin Protein I | AD/Ctrl | PD/Ctrl (15) | |
| 18. | Complement C3c1a | AD/Ctrl | | |
| 19. | Complement Factor H/Hs Protein | AD/Ctrl | PD/Ctrl (17) | |
| 20. | Complement Factor I Protein | AD/Ctrl | PD/Ctrl (4) | AD/PD (14) |
| 21. | Haptoglobin HP-1a Protein | AD/Ctrl | | AD/PD (21) |
| 22. | Similar to Albumin | AD/Ctrl | | |
| 23. | Apolipoprotein A-IV Protein | AD/Ctrl | PD/Ctrl (11) | |
| 24. | Complement Cytolysis Protein | AD/Ctrl | | |
| 25. | Fidgitin Protein II | AD/Ctrl | PD/Ctrl (18) | |
| 26. | Haptoglobin Related Protein | AD/Ctrl | PD/Ctrl (21) | |
| 27. | Alpha-1-microglobulin | AD/Ctrl | | AD/PD (3) |
| 28. | Apolipoprotein E3 Protein | AD/Ctrl | PD/Ctrl (5) | |
| 29. | Int-α-tryp Inh HC (H4) Rel 35 KD Protein | AD/Ctrl | | AD/PD (9) |
| 30. | Glutathione -s-transferase Mu 5-5 | AD/Ctrl | | |
| 31. | Chain A Albumin Mutant R218H Protein | AD/Ctrl | PD/Ctrl (1) | |
| 32. | Apoptosis Inhibitor Scavenger Receptor | AD/Ctrl | | |
| 33. | Albumin Protein | AD/Ctrl | | AD/PD (12) |
| 34. | XI Protein | | PD/Ctrl (3) | AD/PD (26) |
| 35. | Nucleoporin NUP 188 Protein | | PD/Ctrl (7) | AD/PD (23) |
| 36. | Haptoglobin HP-1 Protein | | PD/Ctrl (8) | |
| 37. | Albumin Protein PRO2044 | | PD/Ctrl (9) | |
| 38. | Transthyretin HYPE Protein | | PD/Ctrl (12) | |
| 39. | Chain A Albumin mutant R218H Protein | | PD/Ctrl (14) | AD/PD (11) |
| 40. | Immunoglobulin Kappa Light Chain Protein | | PD/Ctrl(16) | AD/PD (2) |
| 41. | Albumin Protein PRO2675 Protein | | PD/Ctrl (19) | AD/PD (7) |
| 42. | X2 Protein | | PD/Ctrl (20) | AD/PD(10) |
| 43. | Complement C3dg | | | AD/PD (18) |
| 44. | Pre-Apolipoprotein E3 | | | AD/PD (22) |
| 45. | Albumin PR02044 II | | | AD/PD (8) |
| 46. | Immunoglobulin Kappa C Region Protein | | | AD/PD (17) |
| 47. | Hemopexin | | | AD/PD (5) |
| 48. | Complement C3c1b | | | AD/PD (24) |
| 49. | Complement C3c1c | | | AD/PD (25) |
| 50. | Complement Factor Bb | | | AD/PD (1) |
| 51. | Alpha-2-Macroglobulin | | | |
| 52. | Haptoglobin HP-1b Protein | | | |
| 53. | Haptoglobin HP-1e Protein | | | |
| 54. | Complement C3c2a Protein | | | |
| 55. | Transferrin Protein II | | | |
| 56. | X6 Protein | | | |
| 57. | X3 Protein | | | |
| 58. | X4 Pro | | | |

The 47 proteins described for use in the assay of the first embodiment include Apolipoprotein E3, Poly-ADP-ribosyl-A24 (monoubiquitinyl-Histone H2A), Apolipoprotein E4, Apolipoprotein A-IV, Alcohol Dehydrogenase H1A3, Apolipoprotein H, Glutathione-S-Transferase Mu5-5, Clusterin 1, Fidgitin I, Fidgitin II, α-1-microglobulin, α-2-Macroglobulin, Lectin 3 P35, Transthyretin “Dimer”, Pre-serum Amyloid Protein, Haptoglobin HP-1a, Haptoglobin HP-1b, Haptoglobin HP-1c, Haptoglobin HP-1d, Haptoglobin HP-2a Protein, Haptoglobin Related Protein HP-RP, Complement C3c1a, Complement C3c1b, Complement C3c1c, Complement C3c2a, Complement C3c2b, Complement C3dg, Complement C4b Gamma Chain Protein, Complement Factor I, Complement Factor H/Hs, Complement Factor Bb, Complement Cytolysis Protein, Hemopexin, Albumin PRO2044-I, Albumen PRO2675, Inter-α-trypsin Inhibitor Heavy Chain (H4) Related 35 KD Protein, Albumin PRO2044-II, Albumin, Albumin Mutant Chain A R218H-I, Albumin Mutant Chain A R218H-II, Similar to Albumin, Transferrin I, Transferrin II, Immunoglobulin Igxl, and Immunoglobulin Igxc.

### Embodiment 2

A screening/diagnostic assay for drug treated AD patients is also described. Initially the drug treated patients and normal and disease controls are separated into Apo E4 positive and Apo E4 negative groups. By quantitating the serum concentrations of the same 47 proteins as used in the first embodiment, plus an additional 10 serum proteins, the assay can discriminate drug treated patients with Alzheimer's disease (Sensitivity=97.4%) from those with Parkinson's disease and age-matched normal controls (Specificity=95.7%).

The 57 proteins used in the second embodiment are listed in Table 12 and include Apolipoprotein E3, Poly-ADP-ribosyl-A24 (monoubiquitinyl-Histone H2A), Apolipoprotein E4, Apolipoprotein A-IV, Alcohol Dehydrogenase HIA3, Apolipoprotein H, Glutathione-S-Transferase Mu5-5, Clusterin 1, Fidgitin I, Fidgitin II, α-1-microglobulin, α-2-Macroglobulin, Lectin 3 P35, Transthyretin “Dimer”, Pre-serum Amyloid Protein, Haptoglobin HP-1a, Haptoglobin HP-1b, Haptoglobin HP-1c, Haptoglobin HP-1d, Haptoglobin HP-2a Protein, Haptoglobin Related Protein HP-RP, Complement C3c1a, Complement C3c1b, Complement C3c1c, Complement C3c2a, Complement C3c2b, Complement C3dg, Complement C4b Gamma Chain Protein, Complement Factor I, Complement Factor H/Hs, Complement Factor Bb, Complement Cytolysis Protein, Hemopexin, Albumin PRO2044-I, Albumen PRO2675, Inter-a-trypsin Inhibitor Heavy Chain (H4) Related 35 KD Protein, Albumin PRO2044-II, Albumin, Albumin Mutant Chain A R218H-I, Albumin Mutant Chain A R218H-II, Similar to Albumin, Transferrin I, Transferrin II, Immunoglobulin Igxl, and Immunoglobulin Ig_{K}c, Pre-Apolipoprotein E3, Apoptosis Inhibitor AIM-CD5, Nucleoporin 188, Transthyretin Huntingtin Interacting Protein E, and Haptoglobin HP-1e.

The first embodiment provides the basis of a blood test for the differential diagnosis of drug naïve patients useful in the initial diagnosis of Alzheimer's disease, prior to treatment with anti-dementia drugs, by a first physician that suspects that the patient has AD. The second embodiment is more likely to be used by a specialist, such as a neurologist or psychiatrist, for the diagnosis of patients with suspected Alzheimer's disease who are already under treatment by anti dementia drugs, including donazepil, rivastigmine, memantine HCl, or the combination thereof.

The present invention includes a screening assay for neurodegenerative disease based on the up-regulation and/or down-regulation of a first group of proteins involved in cellular degeneration including Apolipoprotein E4, Apolipoprotein E3, and a Transthyretin protein; a second group of Haptoglobin proteins; a third group inflammatory proteins including Complement C3c1, Complement C3c2a protein, Complement C3dg protein, Complement Factor Bb, Complement Factor H, and Inter-alpha Trypsin Inhibitor Heavy Chain (H4); a fourth group of transfer proteins; and a fifth group of proteins of unknown function.

What is claimed is:

1. A method for diagnosing Alzheimer's disease comprising:
collecting a serum sample from a test subject that is untreated with anti-dementia drugs;
analyzing the serum sample for a change in expression of a set of forty seven protein biomarkers; and
using the change in expression of the set of biomarkers to diagnose the test subject.

2. The method of claim 1, wherein the set of biomarkers includes forty-seven serum proteins comprises a first group of proteins involved in cellular degeneration including Apolipoprotein E4, Apolipoprotein E3, and a Transthyretin protein; a second group of Haptoglobin proteins; a third group inflammatory proteins including Complement C3c1, Complement C3c2a protein, Complement C3dg protein, Complement Factor Bb, Complement Factor H, and Inter-alpha Trypsin Inhibitor Heavy Chain (H4); a fourth group of transfer proteins; and a fifth group of proteins of unknown function.

3. A method for diagnosing Alzheimer's disease comprising:
obtaining a patient serum sample from a test subject being treated with an anti-dementia drug;
determining if the patient serum sample contains Apolipoprotein E4 protein; and
quantitating a concentration of a set of 57 serum proteins in the patient serum sample;
whereby a variation in the serum concentration of the set of fifty seven serum proteins from a mean serum concentration of the set of 57 serum protein in non-Alzheimer control serum is a positive diagnosis of Alzheimer's disease.

4. The method of claim 3, wherein the set of 57 serum proteins includes Apolipoprotein E3, Poly-ADP-ribosyl-A24 (monoubiquitinyl-Histone H2A), Apolipoprotein E4, Apolipoprotein A-IV, Alcohol Dehydrogenase H1A3, Apolipoprotein H, Glutathione-S-Transferase Mu5-5, Clusterin 1, Fidgitin I, Fidgitin H, α-1-microglobulin, α-2-Macroglobulin, Lectin 3 P35, Transthyretin “Dimer”, Pre-serum Amyloid Protein, Haptoglobin HP-1a, Haptoglobin HP-1b, Haptoglobin HP-1c, Haptoglobin HP-1d, Haptoglobin HP-2a Protein, Haptoglobin Related Protein HP-RP, Complement C3c1a, Complement C3c1b, Complement C3c1c, Complement C3c2a, Complement C3c2b, Complement C3dg, Complement C4b Gamma Chain Protein, Complement Factor I, Complement Factor H/Hs, Complement Factor Bb, Complement Cytolysis Protein, Hemopexin, Albumin PRO2044-I, Albumen PRO2675, Inter-α-trypsin Inhibitor Heavy Chain (H4) Related 35 KD Protein, Albumin PRO2044-II, Albumin, Albumin Mutant Chain A R218H-I, Albumin Mutant Chain A R218H-II, Similar to Albumin, Transferrin I, Transferrin II, Immunoglobulin Igκl, and Immunoglobulin Igκc, Pre-Apolipoprotein E3, Apoptosis Inhibitor AIM-CD5, Nucleoporin 188, Transthyretin Huntingtin Interacting Protein E, and Haptoglobin HP-1e.
